(19) United States
(12) Patent Application Publication
(10) Pub. No.: US 2023/0310571 A1
(43) Pub. Date: Oct. 5, 2023

(54) HUMAN METAPNEUMOVIRUS VACCINES

(71) Applicant: SANOFI PASTEUR INC., Swiftwater, PA (US)

(72) Inventors: Yvonne Chan, Cambridge, MA (US); Sukanya Sasmal, Cambridge, MA (US); Antonia Stuebler, Cambridge, MA (US); Michael Kishko, Cambridge, MA (US); Sophia Mundle, Cambridge, MA (US); Linong Zhang, Cambridge, MA (US); Josh DiNapoli, Cambridge, MA (US); Judith Alamares-Sapuay, Cambridge, MA (US); Natalie Anosova, Cambridge, MA (US); Sudha Chivukula, Cambridge, MA (US); Hillary Danz, Cambridge, MA (US); Tod Strugnell, Cambridge, MA (US); Rachel Groppo, Cambridge, MA (US)

(21) Appl. No.: 18/070,921

(22) Filed: Nov. 29, 2022

Related U.S. Application Data
(60) Provisional application No. 63/284,405, filed on Nov. 30, 2021.

(51) Int. Cl. A61K 39/12; C07K 14/005; A61K 39/145; A61P 31/14

(52) U.S. Cl. CPC A61K 39/12; C07K 14/005; A61K 39/145; A61P 31/14; C07K 2319/21; C07K 2319/22; A61K 2039/53

(57) ABSTRACT

The present disclosure provides antigenic prefusion hMPV F polypeptides, nucleic acid sequences (e.g., RNA sequences, e.g., mRNA sequences) encoding prefusion hMPV F polypeptides, compositions comprising antigenic prefusion hMPV F polypeptides, compositions comprising nucleic acid sequences encoding prefusion hMPV F polypeptides, and hMPV vaccines.

### RELATED APPLICATIONS

This application claims the benefit of U.S. Provisional Application No. 63/284,405, filed on Nov. 30, 2021, which is incorporated by reference in its entirety for all purposes.

### CRADA STATEMENT

This invention was created in the performance of a Cooperative Research and Development Agreement with the National Institutes of Health, an Agency of the Department of Health and Human Services. The Government of the United States has certain rights in this invention.

### SEQUENCE LISTING

The instant application contains a Sequence Listing which has been submitted electronically in XML format and is hereby incorporated by reference in its entirety. Said XML file, created on Jun. 12, 2023, is named 734287_SA9-325_ST26.xml and is 38,469 bytes in size.

### BACKGROUND OF THE DISCLOSURE

Human metapneumovirus (hMPV) is a leading cause of acute respiratory infection, particularly in children, immunocompromised patients, and the elderly. hMPV, which is closely related to avian metapneumovirus subtype C, has circulated for at least 65 years, and nearly every child will be infected with hMPV by the age of 5. However, immunity is incomplete, and re-infections occur throughout adult life. Symptoms are similar to those of other respiratory viral infections, ranging from mild (e.g., cough, rhinorrhea, and fever) to severe (e.g., bronchiolitis and pneumonia).

There are currently no licensed vaccines or therapeutics against hMPV despite a high disease burden. With the dearth of effective hMPV vaccines or therapeutics available, there exists a need for hMPV vaccines that elicit strong immune responses for potent neutralization of an hMPV infection.

### BRIEF SUMMARY OF THE DISCLOSURE

In one aspect, an antigenic human metapneumovirus (hMPV) prefusion F polypeptide, or a nucleic acid molecule that encodes the same is provided, wherein said prefusion F polypeptide lacks a transmembrane domain and lacks a cytoplasmic tail, and comprises a human rhinovirus 3C (HRV-3C) protease cleavage site.

In certain exemplary embodiments, said prefusion F polypeptide further comprises a F0 cleavage site mutation comprising amino acid substitutions Q100R and S101R, replacing glutamine at amino acid position 100 of SEQ ID NO: 1 with arginine, and replacing serine at amino acid position 101 of SEQ ID NO: 1 with arginine.

In certain exemplary embodiments, said prefusion F polypeptide comprises a signal peptide.

In certain exemplary embodiments, said prefusion F polypeptide comprises at least one tag sequence that is optionally a polyhistidine-tag (e.g., a 6×His tag, 8×His tag, etc.) and/or a Strep II tag.

In certain exemplary embodiments, said prefusion F polypeptide comprises a foldon domain.

In certain exemplary embodiments, said prefusion F polypeptide comprises an amino acid substitution replacing a wild-type amino acid at position 160 of SEQ ID NO: 1, and an amino acid substitution replacing a wild-type amino acid at position 46 of SEQ ID NO: 1.

In certain exemplary embodiments, said prefusion F polypeptide comprises an amino acid substitution replacing threonine at amino acid position 160 of SEQ ID NO: 1, and an amino acid substitution replacing asparagine at amino acid position 46 of SEQ ID NO: 1.

In certain exemplary embodiments, said prefusion F polypeptide comprises an amino acid substitution replacing the amino acid at position 160 with phenylalanine, tryptophan, tyrosine, valine, alanine, isoleucine, or leucine. In certain exemplary embodiments, said prefusion F polypeptide comprises an amino acid substitution replacing the amino acid at position 160 with phenylalanine.

In certain exemplary embodiments, said prefusion F polypeptide comprises an amino acid substitution replacing the amino acid at position 46 with valine, alanine, isoleucine, leucine, phenylalanine, tyrosine, or proline. In certain exemplary embodiments, said prefusion F polypeptide comprises an amino acid substitution replacing the amino acid at position 46 with valine.

In certain exemplary embodiments, the hMPV is A strain or B strain. In certain exemplary embodiments, the hMPV is A1 subtype, A2 subtype, B1 subtype, or B2 subtype.

In certain exemplary embodiments, said prefusion F polypeptide comprises at least 95% sequence identity to SEQ ID NO: 3 or comprises SEQ ID NO: 3.

In certain exemplary embodiments, a messenger RNA (mRNA) comprising an open reading frame (ORF) encoding the F polypeptide is provided.

In certain exemplary embodiments, a method of eliciting an immune response in a subject in need thereof is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a method of preventing an hMPV infection or reducing one or more symptoms of an hMPV infection is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a use of the F polypeptide or nucleic acid molecule, a prophylactically effective amount of the mRNA, or the vaccine, is provided for the manufacture of a medicament for use in treating a subject in need thereof.

In certain exemplary embodiments, the F polypeptide or nucleic acid molecule, a prophylactically effective amount of the mRNA, or the vaccine, is provided for use in treating a subject in need thereof.

In certain exemplary embodiments, a kit comprising a container comprising a single-use or multi-use dosage of the F polypeptide or nucleic acid molecule, a prophylactically effective amount of the mRNA, or the vaccine is provided, optionally wherein the container is a vial or a pre-filled syringe or injector.

In certain exemplary embodiments, an expression vector encoding the F polypeptide, the nucleic acid molecule, or the mRNA is provided.

In certain exemplary embodiments, a cell comprising the expression vector is provided.

In another aspect, an antigenic human metapneumovirus (hMPV) prefusion F polypeptide, or a nucleic acid molecule that encodes the same is provided, wherein said prefusion F polypeptide lacks a transmembrane domain and lacks a cytoplasmic tail, and comprises: an F₀ cleavage site mutation comprising amino acid substitutions Q100R and S101R; replacing glutamine at amino acid position 100 of SEQ ID NO: 1 with arginine, and replacing serine at amino acid position 101 of SEQ ID NO: 1 with arginine; a human rhinovirus 3C (HRV-3C) protease cleavage site; a heterologous signal peptide; a polyhistidine-tag (e.g., a 6×His tag, 8×His tag, etc.) and/or a Strep II tag; and a foldon domain.

In another aspect, an antigenic human prefusion metapneumovirus (hMPV) F polypeptide, or a nucleic acid molecule that encodes the same is provided, wherein said prefusion F polypeptide lacks a transmembrane domain and lacks a cytoplasmic tail, and comprises an amino acid substitution replacing threonine at amino acid position 160 of SEQ ID NO: 1, and an amino acid substitution replacing asparagine at amino acid position 46 of SEQ ID NO: 1.

In certain exemplary embodiments, said prefusion F polypeptide comprises an amino acid substitution replacing threonine at amino acid position 160 with phenylalanine, tryptophan, or tyrosine. In certain exemplary embodiments, said prefusion F polypeptide comprises an amino acid substitution T160F replacing threonine at amino acid position 160 with phenylalanine.

In certain exemplary embodiments, said prefusion F polypeptide comprises an amino acid substitution replacing asparagine at amino acid position 46 with valine, alanine, glycine, isoleucine, leucine, or proline. In certain exemplary embodiments, said prefusion F polypeptide comprises an amino acid substitution N46V replacing asparagine at amino acid position 46 with valine.

In certain exemplary embodiments, said prefusion F polypeptide comprises at least 95% sequence identity to SEQ ID NO: 7.

In certain exemplary embodiments, said prefusion F polypeptide further comprises an F0 cleavage site mutation comprising amino acid substitutions Q100R and S101R, replacing glutamine at amino acid position 100 of SEQ ID NO: 1 with arginine, and replacing serine at amino acid position 101 of SEQ ID NO: 1 with arginine.

In certain exemplary embodiments, said prefusion F polypeptide comprises a signal peptide.

In certain exemplary embodiments, said prefusion F polypeptide comprises at least one tag sequence that is optionally a polyhistidine-tag (e.g., a 6×His tag, 8×His tag, etc.) and/or a Strep II tag.

In certain exemplary embodiments, said prefusion F polypeptide comprises a foldon domain.

In certain exemplary embodiments, the hMPV is A strain or B strain. In certain exemplary embodiments, the hMPV is A1 subtype, A2 subtype, B1 subtype, or B2 subtype.

In certain exemplary embodiments, said prefusion F polypeptide comprises at least 95% sequence identity to SEQ ID NO: 3 or comprises SEQ ID NO: 3.

In certain exemplary embodiments, a messenger RNA (mRNA) comprising an open reading frame (ORF) encoding the F polypeptide is provided.

In certain exemplary embodiments, a method of eliciting an immune response in a subject in need thereof is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a method of preventing an hMPV infection or reducing one or more symptoms of an hMPV infection is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a use of the F polypeptide or nucleic acid molecule, a prophylactically effective amount of the mRNA, or the vaccine, is provided for the manufacture of a medicament for use in treating a subject in need thereof.

In certain exemplary embodiments, the F polypeptide or nucleic acid molecule, a prophylactically effective amount of the mRNA, or the vaccine, is provided for use in treating a subject in need thereof

In certain exemplary embodiments, a kit comprising a container comprising a single-use or multi-use dosage of the F polypeptide or nucleic acid molecule, a prophylactically effective amount of the mRNA, or the vaccine is provided, optionally wherein the container is a vial or a pre-filled syringe or injector.

In certain exemplary embodiments, an expression vector encoding the F polypeptide, the nucleic acid molecule, or the mRNA is provided.

In certain exemplary embodiments, a cell comprising the expression vector is provided.

In another aspect, an antigenic human metapneumovirus (hMPV) prefusion F polypeptide, or a nucleic acid molecule that encodes the same is provided, wherein said prefusion F polypeptide lacks a transmembrane domain and lacks a cytoplasmic tail, and comprises: an amino acid substitution T160F replacing threonine at amino acid position 160 of SEQ ID NO: 1 with phenylalanine, and an amino acid substitution N46V replacing asparagine at amino acid position 46 of SEQ ID NO: 1 with valine; an F₀ cleavage site mutation comprising amino acid substitutions Q100R and S101R; replacing glutamine at amino acid position 100 of SEQ ID NO: 1 with arginine, and replacing serine at amino acid position 101 of SEQ ID NO: 1 with arginine; a human rhinovirus 3C (HRV-3C) protease cleavage site; a signal peptide; a polyhistidine-tag (e.g., a 6×His tag, 8×His tag, etc.) and/or a Strep II tag; and a foldon domain.

In certain exemplary embodiments, the hMPV is A strain or B strain. In certain exemplary embodiments, the hMPV is A1 subtype, A2 subtype, B1 subtype, or B2 subtype.

In certain exemplary embodiments, said prefusion F polypeptide comprises at least 95% sequence identity to SEQ ID NO: 3 or comprises SEQ ID NO: 3.

In certain exemplary embodiments, a messenger RNA (mRNA) comprising an open reading frame (ORF) encoding the F polypeptide is provided.

In certain exemplary embodiments, a method of eliciting an immune response in a subject in need thereof is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a method of preventing an hMPV infection or reducing one or more symptoms of an hMPV infection is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a use of the F polypeptide or nucleic acid molecule, a prophylactically effective amount of the mRNA, or the vaccine, is provided for the manufacture of a medicament for use in treating a subject in need thereof.

In certain exemplary embodiments, the F polypeptide or nucleic acid molecule, a prophylactically effective amount of the mRNA, or the vaccine, is provided for use in treating a subject in need thereof

In certain exemplary embodiments, a kit comprising a container comprising a single-use or multi-use dosage of the F polypeptide or nucleic acid molecule, a prophylactically effective amount of the mRNA, or the vaccine is provided, optionally wherein the container is a vial or a pre-filled syringe or injector.

In certain exemplary embodiments, an expression vector encoding the F polypeptide, the nucleic acid molecule, or the mRNA is provided.

In certain exemplary embodiments, a cell comprising the expression vector is provided.

In another aspect, a human metapneumovirus (hMPV) F polypeptide, or a nucleic acid molecule that encodes the same, wherein said F polypeptide comprises at least 95% sequence identity to SEQ ID NO: 7, is provided.

In certain exemplary embodiments, the F polypeptide is a prefusion F polypeptide.

In certain exemplary embodiments, the F polypeptide is antigenic.

In certain exemplary embodiments, the F polypeptide comprises amino acid substitution T160F replacing threonine at amino acid position 160 with phenylalanine, and amino acid substitution N46V replacing asparagine at amino acid position 46 with valine.

In certain exemplary embodiments, the F polypeptide comprises SEQ ID NO: 7.

In certain exemplary embodiments, a nucleic acid molecule is provided that encodes any of the polypeptides of the above aspect or of any of the above embodiments. In certain exemplary embodiments, the nucleic acid molecule has at least 95% sequence identity to SEQ ID NO: 8. In certain exemplary embodiments, the nucleic acid molecule comprises SEQ ID NO: 8. In certain exemplary embodiments, the nucleic acid molecule has at least 95% sequence identity to SEQ ID NO: 18. In certain exemplary embodiments, the nucleic acid molecule comprises SEQ ID NO: 18. In certain exemplary embodiments, the nucleic acid molecule has at least 95% sequence identity to SEQ ID NO: 19. In certain exemplary embodiments, the nucleic acid molecule comprises SEQ ID NO: 19.

In certain exemplary embodiments, a pharmaceutical composition comprising any of the polypeptides of the above embodiments, or any of the nucleic acid molecules that encode the same, is provided. In certain exemplary embodiments, the pharmaceutical composition is a vaccine.

In certain exemplary embodiments, a method of eliciting an immune response to hMPV or protecting a subject against hMPV infection is provided, comprising administering the vaccine to a subject.

In certain exemplary embodiments, the subject has a comparable serum concentration of neutralizing antibodies against hMPV after administration of the vaccine, relative to a subject that is administered a protein hMPV vaccine. In certain exemplary embodiments, the protein hMPV vaccine is co-administered with an adjuvant.

In certain exemplary embodiments, the vaccine increases the serum concentration of neutralizing antibodies in a subject with pre-existing hMPV immunity.

In certain exemplary embodiments, a vaccine for use in eliciting an immune response to hMPV or protecting a subject against hMPV infection is provided, comprising administering the vaccine to a subject.

In certain exemplary embodiments, the use of the vaccine in the manufacture of a medicament for eliciting an immune response to hMPV or protecting a subject against hMPV infection is provided.

In certain exemplary embodiments, a messenger RNA (mRNA) comprising an open reading frame (ORF) encoding the F polypeptide is provided.

In certain exemplary embodiments, a method of eliciting an immune response in a subject in need thereof is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a method of preventing an hMPV infection or reducing one or more symptoms of an hMPV infection is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a use of the F polypeptide or nucleic acid molecule, a prophylactically effective amount of the mRNA, or the vaccine, is provided for the manufacture of a medicament for use in treating a subject in need thereof.

In certain exemplary embodiments, the F polypeptide or nucleic acid molecule, a prophylactically effective amount of the mRNA, or the vaccine, is provided for use in treating a subject in need thereof

In certain exemplary embodiments, a kit comprising a container comprising a single-use or multi-use dosage of the F polypeptide or nucleic acid molecule, a prophylactically effective amount of the mRNA, or the vaccine is provided, optionally wherein the container is a vial or a pre-filled syringe or injector.

In certain exemplary embodiments, an expression vector encoding the F polypeptide, the nucleic acid molecule, or the mRNA is provided.

In certain exemplary embodiments, a cell comprising the expression vector is provided.

In another aspect, a messenger RNA (mRNA) comprising an open reading frame (ORF) encoding a human metapneumovirus (hMPV) F polypeptide antigen is provided, wherein the hMPV F polypeptide antigen comprises an amino acid sequence with at least 95% identity to SEQ ID NO: 11 or consists of an amino acid sequence of SEQ ID NO: 11.

In certain exemplary embodiments, the hMPV F polypeptide antigen is a pre-fusion F polypeptide.

In certain exemplary embodiments, the ORF is codon optimized.

In certain exemplary embodiments, the mRNA comprises at least one 5′ untranslated region (5′ UTR), at least one 3′ untranslated region (3′ UTR), and at least one polyadenylation (poly(A)) sequence.

In certain exemplary embodiments, the mRNA comprises at least one chemical modification.

In certain exemplary embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In certain exemplary embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

In certain exemplary embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methyluridine, 5-methoxyuridine, and 2′-O-methyl uridine. In certain exemplary embodiments, the chemical modification is selected from the group consisting of pseudouridine, N1-methylpseudouridine, 5-methylcytosine, 5-methoxyuridine, and a combination thereof. In certain exemplary embodiments, the chemical modification is N1-methylpseudouridine.

In certain exemplary embodiments, the mRNA is formulated in a lipid nanoparticle (LNP).

In certain exemplary embodiments, the LNP comprises at least one cationic lipid. In certain exemplary embodiments, the cationic lipid is biodegradable. In certain exemplary embodiments, the cationic lipid is not biodegradable. In certain exemplary embodiments, the cationic lipid is cleavable. In certain exemplary embodiments, the cationic lipid is not cleavable.

In certain exemplary embodiments, the cationic lipid is selected from the group consisting of OF-02, cKK-E10, GL-HEPES-E3-E10-DS-3-E18-1, GL-HEPES-E3-E12-DS-4-E10, and GL-HEPES-E3-E12-DS-3-E14. In certain exemplary embodiments, the cationic lipid is cKK-E10. In certain exemplary embodiments, the cationic lipid is GL-HEPES-E3-E12-DS-4-E10.

In certain exemplary embodiments, the LNP further comprises a polyethylene glycol (PEG) conjugated (PEGylated) lipid, a cholesterol-based lipid, and a helper lipid.

In certain exemplary embodiments, the LNP comprises: a cationic lipid at a molar ratio of 35% to 55%; a polyethylene glycol (PEG) conjugated (PEGylated) lipid at a molar ratio of 0.25% to 2.75%, a cholesterol-based lipid at a molar ratio of 20% to 45%, and a helper lipid at a molar ratio of 5% to 35%, wherein all of the molar ratios are relative to the total lipid content of the LNP.

In certain exemplary embodiments, the LNP comprises: a cationic lipid at a molar ratio of 40%, a PEGylated lipid at a molar ratio of 1.5%, a cholesterol-based lipid at a molar ratio of 28.5%, and a helper lipid at a molar ratio of 30%.

In certain exemplary embodiments, the PEGylated lipid is dimyristoyl-PEG2000 (DMG-PEG2000) or 2-[(polyethylene glycol)-2000]-N,N-ditetradecylacetamide (ALC-0159).

In certain exemplary embodiments, the cholesterol-based lipid is cholesterol.

In certain exemplary embodiments, the helper lipid is 1,2-dioleoyl-SN-glycero-3-phosphoethanolamine (DOPE) or 1,2-distearoyl-sn-glycero-3-phosphocholine (DSPC).

In certain exemplary embodiments, the LNP comprises: GL-HEPES-E3-E12-DS-4-E10 at a molar ratio of 40%, DMG-PEG2000 at a molar ratio of 1.5%, cholesterol at a molar ratio of 28.5%, and DOPE at a molar ratio of 30%.

In certain exemplary embodiments, the LNP comprises: cKK-E10 at a molar ratio of 40%, DMG-PEG2000 at a molar ratio of 1.5%, cholesterol at a molar ratio of 28.5%, and DOPE at a molar ratio of 30%.

In certain exemplary embodiments, the LNP has an average diameter of 30 nm to 200 nm. In certain exemplary embodiments, the LNP has an average diameter of 80 nm to 150 nm.

In certain exemplary embodiments, a pharmaceutical composition is provided comprising the mRNA. In certain exemplary embodiments, the pharmaceutical composition comprises a vaccine.

In certain exemplary embodiments, a method of eliciting an immune response to hMPV or protecting a subject against hMPV infection is provided, comprising administering the vaccine to a subject.

In certain exemplary embodiments, the subject has a comparable serum concentration of neutralizing antibodies against hMPV after administration of the vaccine, relative to a subject that is administered a protein hMPV vaccine. In certain exemplary embodiments, the protein hMPV vaccine is co-administered with an adjuvant.

In certain exemplary embodiments, the vaccine increases the serum concentration of neutralizing antibodies in a subject with pre-existing hMPV immunity.

In certain exemplary embodiments, a vaccine for use in eliciting an immune response to hMPV or protecting a subject against hMPV infection is provided, comprising administering the vaccine to a subject.

In certain exemplary embodiments, the use of the vaccine in the manufacture of a medicament for eliciting an immune response to hMPV or protecting a subject against hMPV infection is provided.

In certain exemplary embodiments, a method of eliciting an immune response in a subject in need thereof is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a method of preventing an hMPV infection or reducing one or more symptoms of an hMPV infection is provided, comprising administering to the subject, optionally intramuscularly, intranasally, intravenously, subcutaneously, or intradermally, a prophylactically effective amount of the F polypeptide or nucleic acid molecule, a prophylactically effective amount of the mRNA, or a prophylactically effective amount of the vaccine.

In certain exemplary embodiments, a use of the F polypeptide or nucleic acid molecule, a prophylactically effective amount of the mRNA, or the vaccine, is provided for the manufacture of a medicament for use in treating a subject in need thereof.

In certain exemplary embodiments, the F polypeptide or nucleic acid molecule, a prophylactically effective amount of the mRNA, or the vaccine, is provided for use in treating a subject in need thereof.

In certain exemplary embodiments, a kit comprising a container comprising a single-use or multi-use dosage of the F polypeptide or nucleic acid molecule, a prophylactically effective amount of the mRNA, or the vaccine is provided, optionally wherein the container is a vial or a pre-filled syringe or injector.

In certain exemplary embodiments, an expression vector encoding the F polypeptide, the nucleic acid molecule, or the mRNA is provided.

In certain exemplary embodiments, a cell comprising the expression vector is provided.

In another aspect, a vaccine is provided comprising a human metapneumovirus (hMPV) F polypeptide antigen or a nucleic acid molecule that encodes the same, wherein the F polypeptide comprises an amino acid sequence having at least 95% identity to SEQ ID NO: 7 or consisting of an amino acid sequence of SEQ ID NO: 7.

In certain exemplary embodiments, the hMPV F polypeptide is a pre-fusion F polypeptide.

In certain exemplary embodiments, a method of eliciting an immune response to hMPV or protecting a subject against hMPV infection is provided, comprising administering the vaccine to a subject.

In certain exemplary embodiments, the vaccine is co-administered with an adjuvant. In certain exemplary embodiments, the vaccine is administered in combination with an additional vaccine. In certain exemplary embodiments, the additional vaccine is a respiratory syncytial virus (RSV) vaccine or an influenza vaccine.

In certain exemplary embodiments, the subject is human. In certain exemplary embodiments, the human subject is an infant, a toddler, or an older adult.

In certain exemplary embodiments, the vaccine increases the serum concentration of neutralizing antibodies, and wherein the subject has pre-existing hMPV immunity.

In certain exemplary embodiments, a vaccine for use in eliciting an immune response to hMPV or protecting a subject against hMPV infection is provided, comprising administering the vaccine to a subject.

In certain exemplary embodiments, a use of the vaccine in the manufacture of a medicament for eliciting an immune response to hMPV or protecting a subject against hMPV infection is provided.

In certain exemplary embodiments, a method of eliciting an immune response in a subject in need thereof, comprising administering to the subject, optionally intramuscularly, intranasally, intravenously, subcutaneously, or intradermally, a prophylactically effective amount of the vaccine is provided.

In certain exemplary embodiments, a method of preventing an hMPV infection or reducing one or more symptoms of an hMPV infection, comprising administering to the subject, optionally intramuscularly, intranasally, intravenously, subcutaneously, or intradermally, a prophylactically effective amount of the vaccine is provided.

In certain exemplary embodiments, a use of the vaccine for the manufacture of a medicament for use in treating a subject in need thereof is provided.

In certain exemplary embodiments, the vaccine is provided for use in treating a subject in need thereof.

In certain exemplary embodiments, a kit comprising a container comprising a single-use or multi-use dosage of the vaccine is provided, optionally wherein the container is a vial or a pre-filled syringe or injector.

In certain exemplary embodiments, an expression vector encoding the F polypeptide, the nucleic acid molecule, or the mRNA is provided.

In certain exemplary embodiments, a cell comprising the expression vector is provided.

### BRIEF DESCRIPTION OF THE DRAWINGS/FIGURES

The foregoing and other features and advantages of the present disclosure will be more fully understood from the following detailed description of illustrative embodiments taken in conjunction with the accompanying drawings. This patent or application file contains at least one drawing executed in color. Copies of this patent or patent application publication with color drawing(s) will be provided by the Office upon request and payment of the necessary fee.

FIG. 1 depicts the design considerations for the panel of 21 candidate hMPV prefusion F antigens shown as two exemplary constructs, D185P and T160_N46V. The construct D185P is used as a benchmark reference to gauge the activities of various novel constructs. “(mut)”=shaded “ENPRRRR(SEQ ID NO: 24)” amino acid sequences and shaded “P”, shaded “V” and shaded “F” singular amino acids; “linker”=bolded, underlined “GGGGS(SEQ ID NO: 25),” “GRS” and “G” amino acid sequences; “foldon”=underlined “GYIPEAPRDGQAYVRKDGEWVLLSTFL” (SEQ ID NO: 26) amino acid sequences; “8×HIS”=shaded “HHHHHHHH (SEQ ID NO: 27)” amino acid sequences; “StrepII”=shaded “SAWSHPQFEK (SEQ ID NO: 28)” sequences.

FIG. 2 depicts the mouse IgG antibody titer against four of the hMPV prefusion F antigen protein constructs measured at day 0, 21, and 35 (data points listed in order from left to right at each time point as follows): (1) A2-F D185P, (2) A2-F T160F_N46V, (3) A2-F K138F, and (4) A2-F G366F_K362F, as well as controls: hMPV (5) A1-F pre-F lot 1, (6) A1-F pre-F lot 2, (7) A1-F post F, and (8) B2 pre-F.

FIG. 3 depicts the mouse hMPV microneutralization antibody titer measured at day 21 and 35 against four of the hMPV prefusion F antigen protein constructs: (1) A2-F D185P, (2) A2-F T160F_N46V, (3) A2-F K138F, and (4) A2-F G366F_K362F as well as controls: hMPV (5) A1 pre-F lot 1, (6) A1 pre-F lot 2, (7) A1 post F, and (8) B2 pre-F.

FIG. 4 depicts the SEC-MALS results for the reference A1 proteins, A1-A185P and A1-postF and the A2 protein antigen candidates, A2-T160F_N46V and A2-D185P.

FIG. 5 depicts representative melting curves [at fluorescence emission 330 and 350 nm] (top panel), smoothened first derivative curve (middle panel), and light scattering [mAU] (bottom panel) for A1-pre-F as well as A1-post-F as measured by nanoDSF.

FIG. 6 depicts a representative melting curve [at fluorescence emission 330 and 350 nm] (top panel) and the smoothened first derivative curve (middle panel), and light scattering [mAU] (bottom panel) for protein samples derived from the A2-D185P and A2-T160F_N46V constructs as measured by nanoDSF.

FIG. 7 depicts the mouse hMPV F antigen IgG antibody titer upon administration of either hMPV prefusion F mRNA constructs, A2-D185P or A2-T160F_N46V, formulated with LNP measured at day 0, 21, and 35.

FIG. 8 depicts the mouse hMPV microneutralization antibody titer upon administration of either hMPV prefusion F mRNA constructs, A2-D185P or A2-T160F_N46V, formulated with LNP measured at day 0, 21, and 35.

FIG. 9 depicts the titers of anti-RSV-F antibodies in mice vaccinated with (1) RSV F mRNA; (2) RSV-F plus hMPV-F mRNA; or (3) RSV protein as measured by end-point ELISA using an RSV-pre-F protein as the binding antigen and detected with rabbit-anti-mouse IgG. Readouts from individual animals (n=8) are shown for the D35 timepoint as log 2 transformed titers with mean+/−95% confidence interval.

FIG. 10 depicts the titers of anti-hMPV-F antibodies in mice vaccinated with (1) hMPV F mRNA; (2) RSV-F plus hMPV-F mRNA; or (3) hMPV protein as measured by end-point ELISA using an hMPV-pre-F protein as the binding antigen and detected with goat-anti-mouse IgG. Readouts from individual animals (n=8) are shown for the D35 timepoint as log 2 transformed titers with mean +1-95% confidence interval.

FIG. 11 depicts RSV neutralizing antibody titers in mice vaccinated with (1) RSV F mRNA; (2) RSV-F plus hMPV-F mRNA; or (3) RSV protein as measured by a microneutralization assay using the RSV A2-GFP strain mixed with serially diluted sera of vaccinated mice on 96-well plates of Vero cells. Titers were determined by calculating the inverse reduction of fluorescent foci after a 24-hour incubation. Readouts from individual animals (n=8) are shown for the D35 timepoint as log 2 transformed titers with mean+/−95% confidence interval.

FIG. 12 depicts hMPV neutralizing antibody titers in mice vaccinated with (1) hMPV F mRNA; (2) RSV-F plus hMPV-F mRNA; or (3) hMPV protein as measured by a microneutralization assay using the hMPV A2-GFP strain mixed with serially diluted sera of vaccinated mice on 96-well plates of Vero cells. Titers were determined by calculating the inverse reduction of fluorescent foci after a 24-hour incubation. Readouts from individual animals (n=8) are shown for the D35 timepoint as log 2 transformed titers with mean+/−95% confidence interval.

FIG. 13 shows an immunoblot of hMPV F protein expression levels for D185P and T160 N46V using 0.3 million cells/well transfected with 1 μg mRNA.

FIG. 14 depicts epitope expression in cells transfected with wild type HMPV F, D185P, or T160F_N46V using pre-F antibody (panel A), post-F antibody (panel B), or a pre-F/post-F antibody (panel C). The top line corresponds to MNR hMPV T160F_N46V, the middle line corresponds to MNR hMPV CAN97-83, and the bottom line corresponds to MNR hMPV D185P in each of panels A, B, and C.

FIG. 15 depicts the hMPV MIMIC setup for evaluating immunogenicity of two hMPV candidates in 24 donors.

FIG. 16 depicts human IgG antibody titer measured at day 14 collected from supernatant of MIMIC co-cultures treated either with IPOL (a polio vaccine) in a 1:50 dilution or an untreated control (no treatment and no human skeletal muscle cells in coculture, “no antigen (w/o HSK)”) to three Polio strains—Polio 1 (panel A), Polio 2 (panel B), and Polio 3 (panel C).

FIG. 17 depicts human IgG antibody titer measured at day 14 collected from supernatant of MIMIC co-cultures treated with 50 ng/ml RSV pre-F NP (RSV pre-F protein fused to ferritin nanoparticles) treatment to RSV pre-F (panel A) and RSV post-F (panel C). Panel C depicts whether the antibodies are functional as measured in an RSV neutralization assay.

FIG. 18 graphically depicts pre-F (panel A) and post-F (panel B) antibody responses. N=22; data presented in Geo Mean with 95% C.I.

FIG. 19 graphically depicts pre-F and post-F neutralizing antibody titers. N=22; data presented in Geo Mean with 95% C.I.

FIG. 20 depicts human IgG antibody titer measured at day 14 collected from supernatant of MIMIC co-cultures treated with experimental groups—hMPV pre-F protein (at 100 ng/ml or 500 ng/ml) or hMPV post F antigen protein (100 ng/ml) or control groups—no antigen w/o HSK, RSV pre-F NP, or IPOL to hMPV pre-F (panel A) or hMPV post-F antigen (panel B).

FIG. 21 depicts a hMPV microneutralization antibody titer measured at day 14 using collected supernatant of MIMIC co-cultures treated with hMPV pre-F protein (at 100 ng/ml or 500 ng/ml), hMPV post F antigen protein (100 ng/ml), or no antigen w/o HSK.

### DETAILED DESCRIPTION OF THE DISCLOSURE

The present disclosure is directed to, inter alia, antigenic prefusion hMPV F polypeptides, nucleic acid sequences (e.g., RNA sequences, e.g., mRNA sequences) encoding antigenic prefusion hMPV F polypeptides, compositions comprising antigenic prefusion hMPV F polypeptides, compositions comprising nucleic acid sequences encoding antigenic prefusion hMPV F polypeptides, and hMPV vaccines.

### I. Definitions

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, protein and nucleic acid chemistry, and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The terms “approximately” or “about” are used herein to mean roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

As used herein, the terms “messenger RNA” or “mRNA” refer to a polynucleotide that encodes at least one polypeptide. mRNA, as used herein, encompasses both modified and unmodified RNA. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a poly(A) tail. mRNA can be purified from natural sources, produced using recombinant expression systems (e.g., in vitro transcription) and optionally purified or chemically synthesized.

As used herein, the term “antigenic site 0” or “site 0 epitope” refers to a site located in the pre-fusion form of the hMPV F trimer. The site 0 epitope is a binding site for antibodies that have specificity for pre-fusion hMPV F.

As used herein, the term “antigenic site V” or “site V epitope” refers to a site located in the pre-fusion form of the hMPV F trimer. The site V epitope is a binding site for antibodies that have specificity for pre-fusion hMPV F.

As used herein, the term “antigen stability” refers to stability of the antigen over time or in solution.

As used herein, the term “cavity filling substitutions” refers to engineered hydrophobic substitutions to fill cavities present in the pre-fusion hMPV F trimer.

As used herein, the term “F protein” or “hMPV F protein” refers to the protein of hMPV responsible for mediating fusion of the viral envelope and the host cell membrane during viral entry. The F protein may mediate fusion between infected cells and non-infected cells to form multinucleated cells or syncytia.

As used herein, the terms “hMPV F polypeptide,” “F polypeptide,” or “F polypeptide antigen” refer to a polypeptide comprising at least one epitope of the hMPV F protein.

As used herein, the term “transmembrane domain” refers to an approximately 23 amino acid sequence near the c-terminus of the hMPV F0/F1 that traverses the membrane of the hMPV virion. In certain embodiments, a transmembrane domain comprises the amino acid sequence

GFIIVIILIAVLGSSMILVSIFII of SEQ ID NO: 22.

As used herein, the term “cytoplasmic tail” refers to an approximately 25 amino acid sequence at the c-terminus of the hMPV F0/F1 that is located inside the virion. In certain embodiments, a transmembrane domain comprises the amino acid sequence

IKKTKKPTGAPPELSGVTNNGFIPHN of SEQ ID NO: 23.

As used herein, a “foldon domain” refers to a trimerization domain of T4 fibritin.

As used herein, a “signal peptide” or “signal sequence” refers to a peptide of approximately 16-30 amino acids in length present at the amino-terminus or the carboxy-terminus of a polypeptide that functions to translocate the polypeptide to the secretory pathway in the endoplasmic reticulum and the Golgi apparatus. In certain embodiments, a signal sequence corresponds to amino acids 1-18 of any one of SEQ ID NO: 1, 3, 5, and 7.

As used herein a “tag sequence” or “affinity tag” refers to a polypeptide sequence that may be used to purify a polypeptide or a protein comprising the tag sequence. Tag sequences include, for example, polyhistidine-tags (e.g., hexahistidine (6×His tag), octahistidine (8×His tag), etc.), glutathione S-transferase (GST), FLAG, streptavidin-binding peptide (SBP), strep II, maltose-binding protein (MBP), calmodulin-binding protein (CBP), chitin-binding domain (CBD), S protein of RNase A, hemagglutinin (HA), c-Myc, and the like.

As used herein, the term “intra-protomer stabilizing substitutions” refers to amino acid substitutions in hMPV F that stabilize the pre-fusion conformation by stabilizing the interaction within a protomer of the hMPV F trimer.

As used herein, the term “inter-protomer stabilizing substitutions” refers to amino acid substitutions in hMPV F that stabilize the pre-fusion conformation by stabilizing the interaction of the protomers of the hMPV F trimer with each other.

As used herein, the term “protease cleavage” refers to proteolysis (sometimes also referred to as “clipping”) of susceptible residues (e.g., lysine or arginine) at a protease cleavage site of a polypeptide sequence. Protease cleavage sites include viral protease cleavage sites such as, e.g., an hMPV F0 protease cleavage site, a respiratory syncytial virus (RSV) F0 protease cleavage site, and a human rhinovirus 3C (HRV-3C) protease cleavage site.

As used herein, the term “post-fusion” with respect to hMPV F refers to a stable conformation of hMPV F that occurs after merging of viral and host cell membranes.

As used herein, the term “pre-fusion” with respect to hMPV F refers to a conformation of hMPV F that is adopted before virus-cell interaction.

As used herein, the term “protomer” refers to a structural unit of an oligomeric protein. In the case of hMPV F, an individual unit of the hMPV F trimer is a protomer.

As used herein, the term “immune response” refers to a response of a cell of the immune system, such as a B cell, T cell, dendritic cell, macrophage, or polymorphonucleocyte, to a stimulus such as an antigen or vaccine. An immune response can include any cell of the body involved in a host defense response, including, for example, an epithelial cell that secretes an interferon or a cytokine. An immune response includes, but is not limited to, an innate and/or adaptive immune response.

As used herein, a “protective immune response” refers to an immune response that protects a subject from infection (e.g., prevents infection or prevents the development of disease associated with infection). Methods of measuring immune responses include measuring, for example, proliferation and/or activity of lymphocytes (such as B or T cells), secretion of cytokines or chemokines, inflammation, antibody production, and the like.

As used herein, an “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity. Antigens include the hMPV polypeptides as described herein.

As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes, killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists and costimulatory molecules.

As used herein, an “antigenic hMPV polypeptide” refers to a polypeptide comprising all or part of an hMPV amino acid sequence of sufficient length that the molecule is antigenic with respect to hMPV.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically engineered animal, and/or a clone. In some embodiments, the terms “individual” or “patient” are used and are intended to be interchangeable with “subject.”

In some embodiments, a “subject” is selected from the group consisting of: a subject aged 65 years old or older, a subject aged 18 to 64 years old (18 to <65 years old), a subject aged 12 years or older, a subject aged 12 to 17 years old (12 to <18 years old), a subject aged 6 to 11 years old (6 to <12 years old), a subject aged 2 to 5 years old (2 to <6 years old), a subject aged 1 to 4 years old (1 to <5 years old), a subject aged 2 months to one year old (2 months to <2 years old), and a subject aged 0 to 2 months old (0 to <3 months old).

In some embodiments, a “subject” is selected from the group consisting of: an older adult (e.g., a senior or elderly adult), an adult, an adolescent, a child, a toddler, and an infant. In some embodiments, a “subject” is selected from the group consisting of: an older adult aged 60 years old or older, an elderly person (e.g., 65 years of age or older), an adult (e.g., 18 to 50 years of age or 18-64 years of age), an adolescent aged 12 to 17 years old (e.g., 12 to <18 years old), a child aged 6 to 11 years old (e.g., 6 to <12 years old), a child aged 2 to 5 years old (e.g., 2 to <6 years old), a toddler aged 1 to 4 years old (e.g., 1 to <5 years old), an infant aged 2 months to 1 year old (e.g., 2 months to <2 years old), a newborn (e.g., 0-27 days of age), and is a preterm newborn infant (e.g., gestational age less than 37 weeks). In some embodiments, a subject is in a pediatric age group as defined by the U.S. FDA: neonate (e.g., birth to less than one month (“NEO”); infant (e.g., age 1 month to less than 2 years (“INF”)); child (e.g., two years to less than 12 years of age (“CHI”)); and adolescent (e.g., ages 12 to less than 17 years (“ADO”)). In some embodiments, a subject is in an older adult in an age group as defined by the U.S. FDA as aged 65 years or older or aged 75 years or older. In particularly exemplary embodiments, a subject is an infant (e.g., age 1 month to less than 2 years), a toddler (e.g., 1 to <5 years old), or an older adult (e.g., aged 60 years or older, 65 years or older, or 75 years or older).

As used herein, the terms “vaccination” or “vaccinate” refer to the administration of a composition intended to generate an immune response, for example, to a disease-causing agent. Vaccination can be administered before, during, and/or after exposure to a disease-causing agent, and/or to the development of one or more symptoms, and in some embodiments, before, during, and/or shortly after exposure to the disease-causing agent. In some embodiments, vaccination includes multiple administrations, appropriately or suitably spaced in time, of a vaccinating composition.

As used herein, the terms “therapeutic” or “therapeutic agent” refer to the administration of a composition intended to lessen or eliminate one or more symptoms of hMPV infection. A therapeutic agent can be administered before, during, and/or after exposure to hMPV, and/or to the development of one or more symptoms. In some embodiments, a therapeutic agent is given to a subject as multiple administrations, appropriately or suitably spaced in time, of a vaccinating composition.

The disclosure describes nucleic acid sequences (e.g., DNA and RNA sequences) and amino acid sequences having a certain degree of identity to a given nucleic acid sequence or amino acid sequence, respectively (e.g., to a reference sequence).

The terms “% identical,” “% identity,” or similar terms are intended to refer, in particular, to the percentage of nucleotides or amino acids which are identical in an optimal alignment between the sequences to be compared. “Sequence identity” between two nucleic acid sequences indicates the percentage of nucleotides that are identical between the sequences. “Sequence identity” between two amino acid sequences indicates the percentage of amino acids that are identical between the sequences. Said percentage is purely statistical, and the differences between the two sequences may be, but are not necessarily, randomly distributed over the entire length of the sequences to be compared. Comparisons of two sequences are usually carried out by comparing said sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments, in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

### II. hMPV F Polypeptide Antigens

Human metapneumovirus (hMPV) is a negative-sense, single-stranded RNA virus belonging to the pneumovirus subfamily within the paramyxovirus family. hMPV infects airway epithelial cells in the nose and lung and is the second most common cause, after respiratory syncytial virus (RSV), of lower respiratory infection in young children. hMPV is an enveloped virus with a glycoprotein (G protein), small hydrophobic protein (SH protein), and a fusion protein (F protein) on the virion surface.

As it is an enveloped virus, entry of hMPV into host cells requires the fusion of viral and cellular membranes. Paramyxovirus entry usually requires two viral glycoproteins, the fusion (F) and attachment (G, H, or HN) proteins, and membrane fusion promoted by all paramyxovirus glycoproteins that have been examined takes place at neutral pH, with one possible exception (i.e., the SER virus). In addition to virus-cell membrane fusion, paramyxovirus glycoproteins also promote cell-cell fusion. Multinucleated giant cells, termed syncytia, can be found in tissues that have been infected by a variety of paramyxoviruses. Cultured cells infected with hMPV form syncytia, but examination of primary human airway epithelial cells infected with hMPV suggests that syncytium formation by this virus may not be a common in vivo occurrence.

hMPV F is a class I fusion glycoprotein synthesized as an inactive precursor (F0) that needs to be cleaved to become fusion competent. Proteolytic cleavage generates two disulfide-linked subunits (F2 N-terminal to F1) that assemble into a homotrimer. Cleavage occurs at a monobasic cleavage site immediately upstream of the hydrophobic fusion peptide. Cleavage can be achieved in tissue culture by addition of exogenous trypsin to the medium or by addition of a furin-expression plasmid. However, in vivo, other serine proteases, such as TMPRSS2, are thought to be likely more relevant for cleavage. The F trimer is incorporated into the virus particle in a metastable, “prefusion” or “pre-F” conformation. To initiate membrane fusion, hMPV F is activated and undergoes a series of stepwise conformational changes in the F protein that drive membrane fusion and result in hMPV F adopting a highly stable “postfusion” or “post-F” conformation.

In certain exemplary embodiments, proteolytic cleavage of F0 is achieved by co-transfection of a plasmid encoding an hMPV F polypeptide and a plasmid encoding furin at a 4:1 ratio hMPV plasmid: furin plasmid.

Provided herein are antigenic hMPV polypeptides comprising an hMPV F polypeptide. The hMPV F polypeptide may comprise the whole sequence of hMPV F or a portion of hMPV F. In certain embodiments, the portion is the ectodomain.

In some embodiments, the hMPV F polypeptide comprises a sequence having at least 80%, 85%, 90%, 95%, 96%, 97%, 98%, 99%, or 99.5% identity to any one of SEQ ID NOs: 1, 3, 5, and 7.

In some embodiments, the hMPV F polypeptide comprises a modified hMPV F polypeptide having at least 80% identity to the polypeptides of any one of SEQ ID NOs: 1, 3, 5, and 7, wherein the hMPV F polypeptide is antigenic.

In some embodiments, the hMPV F polypeptide comprises only the ectodomain portion of the F protein.

The amino acid sequence of F0 for A2-CAN97-83 is:

(SEQ ID NO: 1)
MSWKVVIIFSLLITPQHGLKESYLEESCSTITEGYLSVLRTGWYTNVFT
LEVGDVENLTCSDGPSLIKTELDLTKSALRELKTVSADQLAREEQIENP
RQSRFVLGAIALGVATAAAVTAGVAIAKTIRLESEVTAIKNALKTTNEA
VSTLGNGVRVLATAVRELKDFVSKNLTRAINKNKCDIDDLKMAVSFSQF
NRRFLNVVRQFSDNAGITPAISLDLMTDAELARAVSNMPTSAGQIKLML
ENRAMVRRKGFGILIGVYGSSVIYMVQLPIFGVIDTPCWIVKAAPSCSE
KKGNYACLLREDQGWYCQNAGSTVYYPNEKDCETRGDHVFCDTAAGINV
AEQSKECNINISTTNYPCKVSTGRHPISMVALSPLGALVACYKGVSCSI
GSNRVGIIKQLNKGCSYITNQDADTVTIDNTVYQLSKVEGEQHVIKGRP
VSSSFDPIKFPEDQFNVALDQVFENIENSQALVDQSNRILSSAEKGNTG
FIIVIILIAVLGSSMILVSIFIIIKKTKKPTGAPPELSGVTNNGFIPH
N. (Accession AAN52910; Version AAN52910.1; DB
Source Accession AY145296.1.)

The transmembrane domain is bolded and underlined, and the cytoplasmic tail is bolded.

The nucleotide sequence of F0 for A2-CAN97-83 is:

(SEQ ID NO: 2)
ATGTCTTGGAAAGTGGTGATCATTTTTTCATTGCTAATAACACCTCAA
CACGGTCTTAAAGAGAGCTACCTAGAAGAATCATGTAGCACTATAACT
GAGGGATATCTTAGTGTTCTGAGGACAGGTTGGTATACCAACGTTTTT
ACATTAGAGGTGGGTGATGTAGAAAACCTTACATGTTCTGATGGACCT
AGCCTAATAAAAACAGAATTAGATCTGACCAAAAGTGCACTAAGAGAG
CTCAAAACAGTCTCTGCTGACCAATTGGCAAGAGAGGAACAAATTGAG
AATCCCAGACAATCTAGGTTTGTTCTAGGAGCAATAGCACTCGGTGTT
GCAACAGCAGCTGCAGTCACAGCAGGTGTTGCAATTGCCAAAACCATC
CGGCTTGAGAGTGAAGTCACAGCAATTAAGAATGCCCTCAAAACGACC
AATGAAGCAGTATCTACATTGGGGAATGGAGTTCGAGTGTTGGCAACT
GCAGTGAGAGAGCTGAAAGACTTTGTGAGCAAGAATTTAACTCGTGCA
ATCAACAAAAACAAGTGCGACATTGATGACCTAAAAATGGCCGTTAGC
TTCAGTCAATTCAACAGAAGGTTTCTAAATGTTGTGCGGCAATTTTCA
GACAATGCTGGAATAACACCAGCAATATCTTTGGACTTAATGACAGAT
GCTGAACTAGCCAGGGCCGTTTCTAACATGCCGACATCTGCAGGACAA
ATAAAATTGATGTTGGAGAACCGTGCGATGGTGCGAAGAAAGGGGTTC
GGAATCCTGATAGGGGTCTACGGGAGCTCCGTAATTTACATGGTGCAG
CTGCCAATCTTTGGCGTTATAGACACGCCTTGCTGGATAGTAAAAGCA
GCCCCTTCTTGTTCCGAAAAAAAGGGAAACTATGCTTGCCTCTTAAGA
GAAGACCAAGGGTGGTATTGTCAGAATGCAGGGTCAACTGTTTACTAC
CCAAATGAGAAAGACTGTGAAACAAGAGGAGACCATGTCTTTTGCGAC
ACAGCAGCGGGAATTAATGTTGCTGAGCAATCAAAGGAGTGCAACATC
AACATATCCACTACAAATTACCCATGCAAAGTCAGCACAGGAAGACAT
CCTATCAGTATGGTTGCACTGTCTCCTCTTGGGGCTCTGGTTGCTTGC
TACAAAGGAGTAAGCTGTTCCATTGGCAGCAACAGAGTAGGGATCATC
AAGCAGCTGAACAAGGGTTGCTCCTATATAACCAACCAAGATGCAGAC
ACAGTGACAATAGACAACACTGTATATCAGCTAAGCAAAGTTGAGGGT
GAACAGCATGTTATAAAAGGCAGACCAGTGTCAAGCAGCTTTGATCCA
ATCAAGTTTCCTGAAGATCAATTCAATGTTGCACTTGACCAAGTTTTT
GAGAACATTGAAAACAGCCAGGCCTTGGTAGATCAATCAAACAGAATC
CTAAGCAGTGCAGAGAAAGGGAATACTGGCTTCATCATTGTAATAATT
CTAATTGCTGTCCTTGGCTCTAGCATGATCCTAGTGAGCATCTTCATT
ATAATCAAGAAAACAAAGAAACCAACGGGAGCACCTCCAGAGCTGAGT
GGTGTCACAAACAATGGCTTCATACCACATAATTAG.

In some embodiments, an epitope of the hMPV F protein that is shared between pre-F and post-F is blocked. Blocking an epitope reduces or eliminates the generation of antibodies against the epitope when an RNA (e.g., mRNA) that encodes for the antigenic hMPV F polypeptide is administered to a subject or when an antigenic hMPV F polypeptide is administered to a subject. This can increase the proportion of antibodies that target an epitope specific to a particular conformation of F, such as the pre-fusion conformation (e.g., antibodies that target site 0 and/or site V). Because F has the pre-fusion conformation in viruses that have not yet entered cells, an increased proportion of antibodies that target pre-F can provide a greater degree of neutralization (e.g., expressed as a neutralizing to binding ratio, as described herein).

The hMPV F polypeptides described herein may have deletions or substitutions relative to the wild-type hMPV F protein (e.g., SEQ ID NO: 1).

For example, in certain embodiments, an hMPV polypeptide: (a) lacks a transmembrane domain and lacks a cytoplasmic tail, and comprises a human rhinovirus 3C (HRV-3C) protease cleavage site; (b) comprises a F₀ cleavage site mutation comprising amino acid substitutions Q100R and S101R relative to SEQ ID NO: 1, replacing glutamine at amino acid position 100 with arginine, and replacing serine at amino acid position 101 with arginine; (c) comprises a heterologous signal peptide; (d) comprises at least one tag sequence that is optionally a polyhistidine-tag (e.g., a 6×His tag, 8×His tag, etc.) and/or a Strep II tag; and/or (e) comprises a foldon domain.

In certain embodiments, an hMPV polypeptide lacks a transmembrane domain and lacks a cytoplasmic tail, and comprises: an F0 cleavage site mutation comprising amino acid substitutions Q100R and S101R relative to SEQ ID NO: 1; replacing glutamine at amino acid position 100 with arginine, and replacing serine at amino acid position 101 with arginine; a human rhinovirus 3C (HRV-3C) protease cleavage site; a heterologous signal peptide; a polyhistidine-tag (e.g., a 6×His tag, 8× His tag, etc.) and/or a Strep II tag; and a foldon domain.

In certain embodiments, an hMPV polypeptide includes a valine, alanine, glycine, isoleucine, leucine, or proline substitution at position 185 of SEQ ID NO: 1.

In certain embodiments, an hMPV polypeptide includes a phenylalanine, tryptophan, tyrosine, valine, alanine, isoleucine, or leucine substitution at position 160 of SEQ ID NO: 1, and/or a valine, alanine, isoleucine, leucine, phenylalanine, tyrosine, or proline substitution at position 46 of SEQ ID NO: 1.

In certain embodiments, an hMPV polypeptide includes a substitution at position 160 of SEQ ID NO: 1 and a substitution at position 46 of SEQ ID NO: 1 wherein the substitutions are “stabilizing substitutions” that stabilize the tertiary and/or quaternary structure of an hMPV polypeptide. Stabilizing substitutions include, but are not limited to, substitution of: hydrophobic amino acids (e.g., glycine, alanine, valine, leucine, isoleucine, phenylalanine, tyrosine, tryptophan, proline, and methionine); hydrophilic amino acids (e.g., cysteine, serine, threonine, asparagine, and glutamine; amino acids that forms a disulfide bond (e.g., cysteine); amino acids that form hydrogen bonds (e.g., tryptophan, histidine, tyrosine, and phenylalanine); charged amino acids (e.g., aspartic acid, glutamic acid, arginine, lysine, and histidine), and the like.

In certain embodiments, an hMPV polypeptide is from an A strain hMPV (e.g., an A1 subtype or an A2 subtype) or from a B strain hMPV (e.g., a B1 subtype or a B2 subtype).

In certain embodiments, an amino acid sequence comprising a “backbone” F0 polypeptide sequence is provided, set forth as:

(SEQ ID NO: 3)
MSWKVVIIFSLLITPQHGLKESYLEESCSTITEGYLSVLRTGWYTNV
FTLEVGDVENLTCSDGPSLIKTELDLTKSALRELKTVSADQLAREEQ
IENPRrrRFVLGAIALGVATAAAVTAGVAIAKTIRLESEVTAIKNAL
KTTNEAVSTLGNGVRVLATAVRELKDFVSKNLTRAINKNKCDIDDLK
MAVSFSQFNRRFLNVVRQFSDNAGITPAISLDLMTDAELARAVSNMP
TSAGQIKLMLENRAMVRRKGFGILIGVYGSSVIYMVQLPIFGVIDTP
CWIVKAAPSCSEKKGNYACLLREDQGWYCQNAGSTVYYPNEKDCETR
GDHVFCDTAAGINVAEQSKECNINISTTNYPCKVSTGRHPISMVALS
PLGALVACYKGVSCSIGSNRVGIIKQLNKGCSYITNQDADTVTIDNT
VYQLSKVEGEQHVIKGRPVSSSFDPIKFPEDQFNVALDQVFENIENS
QALVDQSNRILSSAEKGNTggggsgyipeaprdgqayvrkdgewvll
stflgrslevlfqgpghhhhhhhhsawshpqfek.

In certain embodiments, a nucleotide sequence encoding a “backbone” F0 polypeptide sequence is provided, set forth as:

(SEQ ID NO: 4)
ATGAGTTGGAAGGTGGTGATTATCTTCTCCCTGCTGATTACACCACAA
CATGGACTGAAAGAGTCCTACTTGGAGGAGTCCTGTAGCACCATCACA
GAGGGCTACCTGTCTGTGCTGAGGACAGGCTGGTACACCAATGTGTTC
ACCTTGGAGGTGGGAGATGTGGAGAACCTGACTTGTTCTGATGGACCA
TCCCTGATTAAGACAGAACTGGACCTGACCAAGTCTGCCCTGAGGGAA
CTGAAAACAGTGTCTGCTGACCAACTTGCCAGGGAGGAACAGATTGAG
AACCCAAGGAGGAGGAGGTTTGTGCTGGGAGCCATTGCCCTGGGAGTG
GCTACAGCAGCAGCAGTGACAGCAGGAGTGGCTATTGCCAAGACCATC
AGATTGGAGTCTGAGGTGACAGCCATCAAGAATGCCCTGAAAACCACC
AATGAGGCTGTGAGCACCCTGGGCAATGGAGTGAGGGTGCTGGCTACA
GCAGTGAGGGAACTGAAAGACTTTGTGAGCAAGAACCTGACCAGGGCT
ATCAACAAGAACAAGTGTGACATCGATGACCTGAAAATGGCTGTGTCC
TTCAGCCAGTTCAACAGGAGGTTCCTGAATGTGGTGAGACAGTTCTCT
GACAATGCTGGCATCACACCTGCCATCTCCCTGGACCTGATGACAGAT
GCTGAACTGGCAAGGGCTGTGAGCAATATGCCAACCTCTGCTGGACAA
ATCAAACTGATGTTGGAGAACAGGGCTATGGTGAGGAGGAAGGGCTTT
GGCATCCTGATTGGAGTCTATGGCTCCTCTGTGATTTATATGGTCCAA
CTTCCAATCTTTGGAGTGATTGACACACCATGTTGGATTGTGAAGGCT
GCCCCATCCTGTTCTGAGAAGAAGGGCAACTATGCCTGTCTGCTGAGG
GAGGACCAGGGCTGGTATTGTCAGAATGCTGGCAGCACAGTCTACTAC
CCAAATGAGAAGGACTGTGAGACCAGGGGAGACCATGTGTTCTGTGAC
ACAGCAGCAGGCATCAATGTGGCTGAACAGAGCAAGGAGTGTAACATC
AACATCAGCACCACCAACTACCCATGTAAGGTGAGCACAGGCAGACAC
CCAATCAGTATGGTGGCTCTGAGCCCACTGGGAGCCCTGGTGGCTTGT
TACAAGGGAGTGTCCTGTAGCATTGGCAGCAACAGGGTGGGCATCATC
AAGCAACTTAACAAGGGCTGTTCCTACATCACCAACCAGGATGCTGAC
ACAGTGACCATTGACAACACAGTCTACCAACTTAGCAAGGTGGAGGGA
GAACAGCATGTGATTAAGGGCAGACCTGTGTCCTCCTCCTTTGACCCA
ATCAAGTTTCCTGAGGACCAGTTCAATGTGGCTCTGGACCAGGTGTTT
GAGAACATTGAGAACAGCCAGGCTCTGGTGGACCAGAGCAACAGGATT
CTGTCCTCTGCTGAGAAGGGCAACACAGGAGGAGGAGGCTCTGGCTAC
ATCCCTGAGGCTCCAAGGGATGGACAAGCCTATGTGAGGAAGGATGGA
GAGTGGGTGCTGCTGAGCACCTTCCTGGGCAGGTCCTTGGAGGTGCTG
TTCCAGGGACCTGGACACCACCACCACCACCACCACCACTCTGCCTGG
AGCCACCCACAGTTTGAGAAGTAA

In certain embodiments, an hMPV polypeptide comprises a “backbone” hMPV sequence set forth as SEQ ID NO: 3, and may optionally contain one or more amino acid substitutions. For example, in certain embodiments, an hMPV polypeptide includes a valine, alanine, glycine, isoleucine, leucine, or proline substitution at position 185 of SEQ ID NO: 3. In certain embodiments, an hMPV polypeptide includes a phenylalanine, tryptophan, or tyrosine substitution at position 160 of SEQ ID NO: 3, and/or a valine, alanine, glycine, isoleucine, leucine, or proline substitution at position 46 of SEQ ID NO: 3. In certain embodiments, an hMPV polypeptide includes an arginine substitution at one or both of positions 100 and 101 of SEQ ID NO: 3.

In certain embodiments, an hMPV polypeptide has at least 80%, 85%, 90%, 91%, 92%, 93%, 94%, 95%, 96%, 97%, 98%, or 99% sequence identity to SEQ ID NO: 3.

In certain embodiments, an amino acid sequence comprising an hMPV polypeptide sequence is provided, set forth as:

(SEQ ID NO: 5)
MSWKVVIIFSLLITPQHGLKESYLEESCSTITEGYLSVLRTGWYTNV
FTLEVGDVENLTCSDGPSLIKTELDLTKSALRELKTVSADQLAREEQ
IENPRrrRFVLGAIALGVATAAAVTAGVAIAKTIRLESEVTAIKNAL
KTTNEAVSTLGNGVRVLATAVRELKDFVSKNLTRAINKNKCDIpDLK
MAVSFSQFNRRFLNVVRQFSDNAGITPAISLDLMTDAELARAVSNMP
TSAGQIKLMLENRAMVRRKGFGILIGVYGSSVIYMVQLPIFGVIDTP
CWIVKAAPSCSEKKGNYACLLREDQGWYCQNAGSTVYYPNEKDCETR
GDHVFCDTAAGINVAEQSKECNINISTTNYPCKVSTGRHPISMVALS
PLGALVACYKGVSCSIGSNRVGIIKQLNKGCSYITNQDADTVTIDNT
VYQLSKVEGEQHVIKGRPVSSSFDPIKFPEDQFNVALDQVFENIENS
QALVDQSNRILSSAEKGNTggggsgyipeaprdgqayvrkdgewvl
lstflgrslevlfqgpghhhhhhhhsawshpqfek (D185P).

(Lower case amino acids denote a linker, foldon motif, linker, HRV-3C cleavage site, linker, 8×-His-tag, and strep-tag II region.)

In certain embodiments, a nucleotide sequence encoding an hMPV polypeptide sequence is provided, set forth as:

(SEQ ID NO: 6)
ATGAGTTGGAAGGTGGTGATTATCTTCTCCCTGCTGATTACACCACAA
CATGGACTGAAAGAGTCCTACTTGGAGGAGTCCTGTAGCACCATCACA
GAGGGCTACCTGTCTGTGCTGAGGACAGGCTGGTACACCAATGTGTTC
ACCTTGGAGGTGGGAGATGTGGAGAACCTGACTTGTTCTGATGGACCA
TCCCTGATTAAGACAGAACTGGACCTGACCAAGTCTGCCCTGAGGGAA
CTGAAAACAGTGTCTGCTGACCAACTTGCCAGGGAGGAACAGATTGAG
AACCCAAGGAGGAGGAGGTTTGTGCTGGGAGCCATTGCCCTGGGAGTG
GCTACAGCAGCAGCAGTGACAGCAGGAGTGGCTATTGCCAAGACCATC
AGATTGGAGTCTGAGGTGACAGCCATCAAGAATGCCCTGAAAACCACC
AATGAGGCTGTGAGCACCCTGGGCAATGGAGTGAGGGTGCTGGCTACA
GCAGTGAGGGAACTGAAAGACTTTGTGAGCAAGAACCTGACCAGGGCT
ATCAACAAGAACAAGTGTGACATCCCTGACCTGAAAATGGCTGTGTCC
TTCAGCCAGTTCAACAGGAGGTTCCTGAATGTGGTGAGACAGTTCTCT
GACAATGCTGGCATCACACCTGCCATCTCCCTGGACCTGATGACAGAT
GCTGAACTGGCAAGGGCTGTGAGCAATATGCCAACCTCTGCTGGACAA
ATCAAACTGATGTTGGAGAACAGGGCTATGGTGAGGAGGAAGGGCTTT
GGCATCCTGATTGGAGTCTATGGCTCCTCTGTGATTTATATGGTCCAA
CTTCCAATCTTTGGAGTGATTGACACACCATGTTGGATTGTGAAGGCT
GCCCCATCCTGTTCTGAGAAGAAGGGCAACTATGCCTGTCTGCTGAGG
GAGGACCAGGGCTGGTATTGTCAGAATGCTGGCAGCACAGTCTACTAC
CCAAATGAGAAGGACTGTGAGACCAGGGGAGACCATGTGTTCTGTGAC
ACAGCAGCAGGCATCAATGTGGCTGAACAGAGCAAGGAGTGTAACATC
AACATCAGCACCACCAACTACCCATGTAAGGTGAGCACAGGCAGACAC
CCAATCAGTATGGTGGCTCTGAGCCCACTGGGAGCCCTGGTGGCTTGT
TACAAGGGAGTGTCCTGTAGCATTGGCAGCAACAGGGTGGGCATCATC
AAGCAACTTAACAAGGGCTGTTCCTACATCACCAACCAGGATGCTGAC
ACAGTGACCATTGACAACACAGTCTACCAACTTAGCAAGGTGGAGGGA
GAACAGCATGTGATTAAGGGCAGACCTGTGTCCTCCTCCTTTGACCCA
ATCAAGTTTCCTGAGGACCAGTTCAATGTGGCTCTGGACCAGGTGTTT
GAGAACATTGAGAACAGCCAGGCTCTGGTGGACCAGAGCAACAGGATT
CTGTCCTCTGCTGAGAAGGGCAACACAGGAGGAGGAGGCTCTGGCTAC
ATCCCTGAGGCTCCAAGGGATGGACAAGCCTATGTGAGGAAGGATGGA
GAGTGGGTGCTGCTGAGCACCTTCCTGGGCAGGTCCTTGGAGGTGCTG
TTCCAGGGACCTGGACACCACCACCACCACCACCACCACTCTGCCTGG
AGCCACCCACAGTTTGAGAAGTAA (D185P).

In certain embodiments, a nucleotide sequence encoding an hMPV polypeptide sequence is provided, set forth as:

(SEQ ID NO: 17)
ATGTCTTGGAAAGTCGTCATCATCTTCTCTCTGCTGATCACCCCACAA
CACGGCCTGAAGGAATCTTATCTGGAAGAGTCCTGCTCCACAATCACA
GAGGGCTACCTGAGCGTGCTGAGAACCGGCTGGTACACCAACGTGTTC
ACTCTGGAGGTGGGCGACGTGGAGAACCTGACTTGTAGTGACGGCCCC
TCCCTGATCAAGACTGAGCTGGACCTGACAAAGAGTGCACTGAGAGAA
CTCAAGACTGTGTCCGCAGACCAGCTGGCCCGCGAGGAGCAGATCGAA
AATCCTAGACAGTCAAGGTTCGTCCTGGGAGCCATTGCTCTGGGAGTT
GCTACAGCTGCCGCTGTGACCGCAGGGGTGGCTATTGCTAAAACCATC
AGGCTGGAGTCCGAAGTGACAGCAATCAAGAATGCCCTGAAGACCACC
AACGAGGCAGTCTCCACACTGGGCAATGGAGTGAGGGTGCTGGCAACC
GCCGTGAGGGAGCTGAAGGACTTCGTGTCCAAGAACCTGACCAGGGCT
ATCAACAAAAACAAGTGCGACATCCCCGATCTGAAGATGGCAGTTAGC
TTTTCCCAGTTTAACCGGAGATTCCTGAATGTGGTTAGACAGTTCAGC
GACAACGCCGGGATCACCCCAGCTATTTCCCTGGACCTGATGACTGAT
GCCGAGCTGGCACGGGCTGTGTCCAATATGCCCACCAGCGCTGGGCAG
ATTAAGCTGATGCTGGAGAATCGGGCAATGGTGAGAAGGAAGGGGTTT
GGCATCCTGATCGGCGTGTACGGGTCCTCCGTGATCTACATGGTGCAG
CTGCCTATTTTTGGAGTGATTGATACACCCTGCTGGATCGTTAAAGCA
GCACCCAGCTGCTCCGAGAAGAAGGGCAATTACGCCTGTCTGCTGCGG
GAGGACCAGGGGTGGTACTGCCAGAACGCCGGCTCCACAGTGTATTAC
CCCAATGAAAAGGACTGCGAGACAAGGGGAGACCACGTGTTCTGCGAC
ACTGCCGCTGGGATTAATGTGGCCGAGCAGAGCAAGGAGTGCAACATC
AACATTTCCACCACAAACTACCCCTGCAAGGTGAGCACCGGCAGGCAC
CCTATCTCCATGGTGGCCCTGTCTCCCCTGGGAGCTCTGGTGGCTTGC
TACAAGGGAGTGAGCTGTAGCATCGGGTCCAATAGAGTCGGGATTATC
AAGCAGCTGAATAAGGGCTGCAGCTATATTACCAACCAGGATGCCGAT
ACTGTGACTATTGACAACACAGTGTATCAGCTGTCAAAGGTGGAAGGC
GAACAGCATGTGATCAAAGGACGGCCCGTCAGCAGCTCCTTTGACCCT
ATCAAATTCCCCGAAGACCAGTTTAACGTGGCACTGGACCAGGTTTTC
GAAAATATTGAGAATTCTCAGGCCCTGGTGGACCAGTCTAACCGGATC
CTCTCCTCCGCCGAGAAGGGAAATACAGGCTTTATTATCGTGATCATC
CTGATCGCAGTGCTGGGATCCAGTATGATCCTGGTCTCCATTTTCATC
ATCATTAAGAAGACCAAGAAACCCACTGGCGCACCACCTGAACTGAGC
GGCGTGACTAACAATGGCTTTATCCCTCACAATTGA
(D185P mRNA).

In certain embodiments, an hMPV polypeptide has at least 80%, 85%, 90%, 91%, 92%, 93%, 94%, 95%, 96%, 97%, 98%, or 99% sequence identity to SEQ ID NO: 5. In certain embodiments, an hMPV polypeptide comprises SEQ ID NO: 5. In certain embodiments, an hMPV polynucleotide has at least 80%, 85%, 90%, 91%, 92%, 93%, 94%, 95%, 96%, 97%, 98%, or 99% sequence identity to SEQ ID NO: 6. In certain embodiments, an hMPV polynucleotide comprises SEQ ID NO: 6. In certain embodiments, an hMPV polynucleotide has at least 80%, 85%, 90%, 91%, 92%, 93%, 94%, 95%, 96%, 97%, 98%, or 99% sequence identity to SEQ ID NO: 17. In certain embodiments, an hMPV polynucleotide comprises SEQ ID NO: 17.

In certain embodiments, an amino acid sequence comprising an hMPV polypeptide sequence is provided, set forth as:

(SEQ ID NO: 7)
MSWKVVIIFSLLITPQHGLKESYLEESCSTITEGYLSVLRTGWYTVVF
TLEVGDVENLTCSDGPSLIKTELDLTKSALRELKTVSADQLAREEQIE
NPRrrRFVLGAIALGVATAAAVTAGVAIAKTIRLESEVTAIKNALKTT
NEAVSTLGNGVRVLAfAVRELKDFVSKNLTRAINKNKCDIDDLKMAVS
FSQFNRRFLNVVRQFSDNAGITPAISLDLMTDAELARAVSNMPTSAGQ
IKLMLENRAMVRRKGFGILIGVYGSSVIYMVQLPIFGVIDTPCWIVKA
APSCSEKKGNYACLLREDQGWYCQNAGSTVYYPNEKDCETRGDHVFCD
TAAGINVAEQSKECNINISTTNYPCKVSTGRHPISMVALSPLGALVAC
YKGVSCSIGSNRVGIIKQLNKGCSYITNQDADTVTIDNTVYQLSKVEG
EQHVIKGRPVSSSFDPIKFPEDQFNVALDQVFENIENSQALVDQSNRI
LSSAEKGNTggggsgyipeaprdgqayvrkdgewvllstflgrslevl
fqgpghhhhhhhhsawshpqfek (T160F_N46V).

(Lower case amino acids denote a linker, foldon motif, linker, HRV-3C cleavage site, linker, 8×-His-tag, and strep-tag II region.)

In certain embodiments, a nucleotide sequence encoding an hMPV polypeptide sequence is provided, set forth as:

(SEQ ID NO: 8)
ATGAGTTGGAAGGTGGTGATTATCTTCTCCCTGCTGATTACACCACAA
CATGGACTGAAAGAGTCCTACTTGGAGGAGTCCTGTAGCACCATCACA
GAGGGCTACCTGTCTGTGCTGAGGACAGGCTGGTACACAGTGGTGTTC
ACCTTGGAGGTGGGAGATGTGGAGAACCTGACTTGTTCTGATGGACCA
TCCCTGATTAAGACAGAACTGGACCTGACCAAGTCTGCCCTGAGGGAA
CTGAAAACAGTGTCTGCTGACCAACTTGCCAGGGAGGAACAGATTGAG
AACCCAAGGAGGAGGAGGTTTGTGCTGGGAGCCATTGCCCTGGGAGTG
GCTACAGCAGCAGCAGTGACAGCAGGAGTGGCTATTGCCAAGACCATC
AGATTGGAGTCTGAGGTGACAGCCATCAAGAATGCCCTGAAAACCACC
AATGAGGCTGTGAGCACCCTGGGCAATGGAGTGAGGGTGCTGGCTTTT
GCTGTGAGGGAACTGAAAGACTTTGTGAGCAAGAACCTGACCAGGGCT
ATCAACAAGAACAAGTGTGACATTGATGACCTGAAAATGGCTGTGTCC
TTCAGCCAGTTCAACAGGAGGTTCCTGAATGTGGTGAGACAGTTCTCT
GACAATGCTGGCATCACACCTGCCATCTCCCTGGACCTGATGACAGAT
GCTGAACTGGCAAGGGCTGTGAGCAATATGCCAACCTCTGCTGGACAA
ATCAAACTGATGTTGGAGAACAGGGCTATGGTGAGGAGGAAGGGCTTT
GGCATCCTGATTGGAGTCTATGGCTCCTCTGTGATTTATATGGTCCAA
CTTCCAATCTTTGGAGTGATTGACACACCATGTTGGATTGTGAAGGCT
GCCCCATCCTGTTCTGAGAAGAAGGGCAACTATGCCTGTCTGCTGAGG
GAGGACCAGGGCTGGTATTGTCAGAATGCTGGCAGCACAGTCTACTAC
CCAAATGAGAAGGACTGTGAGACCAGGGGAGACCATGTGTTCTGTGAC
ACAGCAGCAGGCATCAATGTGGCTGAACAGAGCAAGGAGTGTAACATC
AACATCAGCACCACCAACTACCCATGTAAGGTGAGCACAGGCAGACAC
CCAATCAGTATGGTGGCTCTGAGCCCACTGGGAGCCCTGGTGGCTTGT
TACAAGGGAGTGTCCTGTAGCATTGGCAGCAACAGGGTGGGCATCATC
AAGCAACTTAACAAGGGCTGTTCCTACATCACCAACCAGGATGCTGAC
ACAGTGACCATTGACAACACAGTCTACCAACTTAGCAAGGTGGAGGGA
GAACAGCATGTGATTAAGGGCAGACCTGTGTCCTCCTCCTTTGACCCA
ATCAAGTTTCCTGAGGACCAGTTCAATGTGGCTCTGGACCAGGTGTTT
GAGAACATTGAGAACAGCCAGGCTCTGGTGGACCAGAGCAACAGGATT
CTGTCCTCTGCTGAGAAGGGCAACACAGGAGGAGGAGGCTCTGGCTAC
ATCCCTGAGGCTCCAAGGGATGGACAAGCCTATGTGAGGAAGGATGGA
GAGTGGGTGCTGCTGAGCACCTTCCTGGGCAGGTCCTTGGAGGTGCTG
TTCCAGGGACCTGGACACCACCACCACCACCACCACCACTCTGCCTGG
AGCCACCCACAGTTTGAGAAGTAA (T160F_N46V).

In certain embodiments, a nucleotide sequence encoding an hMPV polypeptide sequence is provided, set forth as:

(SEQ ID NO: 18)
ATGAGCTGGAAGGTTGTGATTATTTTCTCTCTGCTGATTACTCCACAG
CACGGCCTGAAGGAGTCCTACCTGGAGGAGTCCTGTTCTACTATCACT
GAGGGGTATCTCTCTGTGCTGCGGACAGGGTGGTATACAGTGGTGTTC
ACCCTGGAGGTTGGCGATGTGGAGAATCTGACTTGCAGCGATGGCCCT
TCTCTGATCAAGACCGAGCTGGATCTGACAAAAAGCGCCCTCAGAGAA
CTGAAAACCGTGTCCGCCGATCAGCTGGCAAGGGAGGAGCAGATCGAG
AACCCACGGCAGAGCAGGTTTGTGCTGGGCGCTATCGCTCTGGGCGTG
GCCACTGCAGCTGCTGTCACTGCAGGGGTCGCAATCGCTAAGACTATC
AGACTGGAATCCGAGGTGACCGCCATTAAGAATGCCCTGAAGACTACC
AACGAGGCTGTGTCCACTCTGGGAAACGGAGTGAGGGTCCTGGCCTTC
GCAGTGAGGGAGCTGAAGGATTTTGTGTCAAAGAACCTTACACGGGCC
ATCAACAAGAATAAGTGCGATATCGATGACCTGAAGATGGCCGTGTCC
TTCTCCCAGTTCAACCGGCGCTTTCTGAATGTGGTGCGCCAGTTTTCC
GACAACGCTGGAATCACCCCTGCTATCAGCCTGGACCTCATGACCGAC
GCCGAACTCGCAAGGGCCGTTTCTAACATGCCTACATCCGCTGGACAG
ATTAAGCTGATGCTGGAGAATAGAGCAATGGTGAGGAGAAAGGGATTC
GGCATCCTGATTGGCGTGTACGGATCTAGCGTGATCTACATGGTGCAG
CTGCCGATCTTCGGCGTGATCGATACTCCTTGTTGGATCGTCAAGGCC
GCCCCTTCCTGCTCCGAGAAGAAGGGCAATTACGCTTGTCTGCTGCGG
GAGGACCAGGGCTGGTATTGCCAGAACGCCGGGTCTACAGTGTACTAT
CCTAACGAGAAGGATTGCGAGACCAGAGGCGACCACGTTTTCTGTGAT
ACAGCCGCCGGAATCAATGTCGCAGAGCAGTCTAAGGAGTGCAACATC
AATATCTCTACAACCAATTACCCATGTAAGGTGAGCACTGGACGGCAC
CCTATCAGTATGGTGGCTCTGAGCCCACTGGGGGCACTGGTGGCTTGC
TACAAGGGGGTGAGCTGCAGTATCGGCAGTAACAGAGTGGGCATTATC
AAGCAGCTGAACAAAGGGTGCTCTTATATTACAAACCAGGATGCAGAT
ACTGTGACCATCGACAACACTGTGTACCAGCTGTCCAAGGTGGAGGGG
GAGCAGCATGTGATCAAAGGGAGACCCGTCTCTTCTTCTTTCGATCCC
ATCAAGTTCCCTGAAGACCAGTTCAATGTTGCCCTGGACCAGGTTTTC
GAGAACATCGAAAATAGCCAGGCCTTGGTCGATCAATCCAACAGGATC
CTGAGCAGCGCAGAGAAAGGGAACACTGGCTTCATCATCGTGATCATT
CTGATCGCCGTGCTGGGGAGCAGTATGATTCTGGTGTCCATTTTCATC
ATCATCAAGAAGACCAAGAAGCCTACAGGAGCACCCCCTGAGCTGAGC
GGAGTGACCAACAACGGCTTTATCCCTCACAACTGA
(T160F_N46V).

In certain embodiments, a nucleotide sequence encoding an hMPV polypeptide sequence is provided, set forth as:

(SEQ ID NO: 19)
ATGAGCTGGAAGGTTGTGATTATTTTCTCTCTGCTGATTACTCCACAG
CACGGCCTGAAGGAGTCCTACCTGGAGGAGTCCTGTTCTACTATCACT
GAGGGGTATCTCTCTGTGCTGCGGACAGGGTGGTATACAGTGGTGTTC
ACCCTGGAGGTTGGCGATGTGGAGAATCTGACTTGCAGCGATGGCCCT
TCTCTGATCAAGACCGAGCTGGATCTGACAAAAAGCGCCCTCAGAGAA
CTGAAAACCGTGTCCGCCGATCAGCTGGCAAGGGAGGAGCAGATCGAG
AACCCACGGCAGAGCAGGTTTGTGCTGGGCGCTATCGCTCTGGGCGTG
GCCACTGCAGCTGCTGTCACTGCAGGGGTCGCAATCGCTAAGACTATC
AGACTGGAATCCGAGGTGACCGCCATTAAGAATGCCCTGAAGACTACC
AACGAGGCTGTGTCCACTCTGGGAAACGGAGTGAGGGTCCTGGCCTTC
GCAGTGAGGGAGCTGAAGGATTTTGTGTCAAAGAACCTTACACGGGCC
ATCAACAAGAATAAGTGCGATATCGATGACCTGAAGATGGCCGTGTCC
TTCTCCCAGTTCAACCGGCGCTTTCTGAATGTGGTGCGCCAGTTTTCC
GACAACGCTGGAATCACCCCTGCTATCAGCCTGGACCTCATGACCGAC
GCCGAACTCGCAAGGGCCGTTTCTAACATGCCTACATCCGCTGGACAG
ATTAAGCTGATGCTGGAGAATAGAGCAATGGTGAGGAGAAAGGGATTC
GGCATCCTGATTGGCGTGTACGGATCTAGCGTGATCTACATGGTGCAG
CTGCCGATCTTCGGCGTGATCGATACTCCTTGTTGGATCGTCAAGGCC
GCCCCTTCCTGCTCCGAGAAGAAGGGCAATTACGCTTGTCTGCTGCGG
GAGGACCAGGGCTGGTATTGCCAGAACGCCGGGTCTACAGTGTACTAT
CCTAACGAGAAGGATTGCGAGACCAGAGGCGACCACGTTTTCTGTGAT
ACAGCCGCCGGAATCAATGTCGCAGAGCAGTCTAAGGAGTGCAACATC
AATATCTCTACAACCAATTACCCATGTAAGGTGAGCACTGGACGGCAC
CCTATCAGTATGGTGGCTCTGAGCCCACTGGGGGCACTGGTGGCTTGC
TACAAGGGGGTGAGCTGCAGTATCGGCAGTAACAGAGTGGGCATTATC
AAGCAGCTGAACAAAGGGTGCTCTTATATTACAAACCAGGATGCAGAT
ACTGTGACCATCGACAACACTGTGTACCAGCTGTCCAAGGTGGAGGGG
GAGCAGCATGTGATCAAAGGGAGACCCGTCTCTTCTTCTTTCGATCCC
ATCAAGTTCCCTGAAGACCAGTTCAATGTTGCCCTGGACCAGGTTTTC
GAGAACATCGAAAATAGCCAGGCCTTGGTCGATCAATCCAACAGGATC
CTGAGCAGCGCAGAGAAAGGGAACACTGGCTTCATCATCGTGATCATT
CTGATCGCCGTGCTGGGGAGCAGTATGATTCTGGTGTCCATTTTCATC
ATCATCAAGAAGACCAAGAAGCCTACAGGAGCACCCCCTGAGCTGAGC
GGAGTGACCAACAACGGCTTTATCCCTCACAACTAA
(T160F_N46V).

In certain embodiments, an hMPV polypeptide has at least 80%, 85%, 90%, 91%, 92%, 93%, 94%, 95%, 96%, 97%, 98%, or 99% sequence identity to SEQ ID NO: 7. In certain embodiments, an hMPV polypeptide comprises SEQ ID NO: 7. In certain embodiments, an hMPV polynucleotide has at least 80%, 85%, 90%, 91%, 92%, 93%, 94%, 95%, 96%, 97%, 98%, or 99% sequence identity to SEQ ID NO: 8. In certain embodiments, an hMPV polynucleotide comprises SEQ ID NO: 8. In certain embodiments, an hMPV polynucleotide has at least 80%, 85%, 90%, 91%, 92%, 93%, 94%, 95%, 96%, 97%, 98%, or 99% sequence identity to SEQ ID NO: 18. In certain embodiments, an hMPV polynucleotide comprises SEQ ID NO: 18. In certain embodiments, an hMPV polynucleotide has at least 80%, 85%, 90%, 91%, 92%, 93%, 94%, 95%, 96%, 97%, 98%, or 99% sequence identity to SEQ ID NO: 8. In certain embodiments, an hMPV polynucleotide comprises SEQ ID NO: 8. In certain embodiments, an hMPV polynucleotide has at least 80%, 85%, 90%, 91%, 92%, 93%, 94%, 95%, 96%, 97%, 98%, or 99% sequence identity to SEQ ID NO: 19. In certain embodiments, an hMPV polynucleotide comprises SEQ ID NO: 19.

In general, positions in constructs described herein can be mapped onto a reference sequence, e.g., the wild-type sequence of SEQ ID NO: 1 or the backbone sequence of SEQ ID NO: 3, by pairwise alignment, e.g., using the Needleman-Wunsch algorithm with standard parameters (EBLOSUM62 matrix, Gap penalty 10, gap extension penalty 0.5).

### III. Recombinant hMPV F Polypeptide Antigens

In certain embodiments, hMPV vaccines of the present disclosure may comprise at least one hMPV F polypeptide antigen. hMPV F polypeptide antigens of the disclosure can be made by a variety of methods. In one embodiment, a host cell line that can be of eukaryotic or prokaryotic origin is used for expression of an hMPV F polypeptide. In one embodiment, a host cell line used for expression of an hMPV F polypeptide is of bacterial origin. In one embodiment, a host cell line used for expression of an hMPV F polypeptide is of mammalian origin. Particular host cell lines which are best suited for the desired gene product to be expressed therein can be determined. Exemplary host cell lines include, but are not limited to, DG44 and DUXB11 (Chinese hamster ovary lines, DHFR minus), HELA (human cervical carcinoma), CVI (monkey kidney line), COS (a derivative of CVI with SV40 T antigen), CHO (Chinese hamster ovary), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), and 293 (human kidney). Host cell lines are typically available from commercial services, the American Tissue Culture Collection (ATCC) or from published literature.

In certain embodiments, baculovirus cells may be used to express an hMPV F polypeptide antigen described herein. The baculovirus Autographa californica Nuclear Polyhedrosis Virus (AcNPV), for example, can be used to express an hMPV F polypeptide.

A recombinant baculovirus may be constructed to express hMPV F polypeptide by homologous recombination between baculovirus DNA and chimeric plasmids containing the hMPV F sequence of interest. Recombinant viruses can be detected by virtue of their distinct plaque morphology and plaque-purified to homogeneity.

Recombinant hMPV F polypeptides can be produced in cells that include, but are not limited to, cells derived from the Lepidopteran species Spodoptera frugiperda. Other suitable insect cells that can be infected by baculovirus, such as those from the species Bombyx mori, Galleria mellanoma, Trichplusia ni, or Lamanthria dispar, could also be used as a suitable substrate to produce recombinant hMPV F polypeptide.

Recombinant hMPV F polypeptide can also be expressed in other expression vectors such as Entomopox viruses (the poxviruses of insects), cytoplasmic polyhedrosis viruses (CPV), and transformation of insect cells with the recombinant hMPV F gene constitutive expression.

Baculovirus expression of recombinant proteins is described further in U.S. Pat. No. 5,762,919, incorporated herein by reference in its entirety for all purposes.

In certain embodiments, algal cells, e.g., microalgal cells, can be used to express a recombinant hMPV F polypeptide antigen as described herein. In some embodiments, the microalgal host cell is a heterokont or stramenopile. In some embodiments, the microalgal host cell is a member of the phylum Labyrinthulomycota. In some embodiments, the Labyrinthulomycota host cell is a member of the order Thraustochytriales or the order Labyrinthulales.

The expression system used for expression of an hMPV polypeptide antigen in a microalgal host cell comprises regulatory control elements that are active in microalgal cells. In some embodiments, the expression system comprises regulatory control elements that are active in Labyrinthulomycota cells. In some embodiments, the expression system comprises regulatory control elements that are active in thraustochytrids. In some embodiments, the expression system comprises regulatory control elements that are active in Schizochytrium or Thraustochytrium. Many regulatory control elements, including various promoters, are active in a number of diverse species. Therefore, regulatory sequences can be utilized in a cell type that is identical to the cell from which they were isolated or can be utilized in a cell type that is different than the cell from which they were isolated.

In some embodiments, the expression system used for hMPV F polypeptide production in microalgal cells comprises regulatory elements that are derived from Labyrinthulomycota sequences. In some embodiments, the expression system used to produce hMPV F polypeptides in microalgal cells comprises regulatory elements that are derived from non-Labyrinthulomycota sequences, including sequences derived from non-Labyrinthulomycota algal sequences. In some embodiments, the expression system comprises a polynucleotide sequence encoding an hMPV F polypeptide, wherein the polynucleotide sequence is associated with any promoter sequence, any terminator sequence, and/or any other regulatory sequences that are functional in a microalgal host cell. Inducible or constitutively active sequences can be used. In certain embodiments, an expression cassette for expression of an hMPV F polypeptide in a microalgal host cell is provided as well as algal cells comprising the same.

Microalgal expression of recombinant proteins is described further in International Pub. Nos. WO 2011/082189 and WO 2011/090731, incorporated herein by reference in their entireties for all purposes.

In certain embodiments, CHO cells may be used to express an hMPV F polypeptide described herein. In certain embodiments, a CHO cell line comprising a vector expressing hMPV F is provided. In certain embodiments, said CHO cell line is transfected (stably or transiently transfected) with said vector. In certain embodiments, said CHO cell line comprises said vector integrated in its genome. CHO cell lines are commonly used for industrial protein production, and many CHO cell lines are known and are commercially available, e.g., from ATCC. For instance, such CHO cell lines include, e.g., the CHO-K1 cell line (ATCC Number: CCL-61), the CHO DP-12 cell line (ATCC Nos. CRL-12444 and 12445), and the CHO 1-15 cell line (ATCC Number CRL-9606).

In vitro production allows scale-up to give large amounts of the desired polypeptides. Techniques for cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified using customary chromatography methods, for example, gel filtration, ion-exchange chromatography, chromatography over DEAE-cellulose, and/or (immuno-) affinity chromatography.

### IV. RNA

In certain embodiments, hMPV vaccines of the present disclosure may comprise at least one ribonucleic acid (RNA) comprising an ORF encoding an hMPV F polypeptide antigen. In certain embodiments, the RNA is a messenger RNA (mRNA) comprising an ORF encoding an hMPV F protein antigen. In certain embodiments, the RNA (e.g., mRNA) further comprises at least one 5′ UTR, 3′ UTR, a poly(A) tail, and/or a 5′ cap.

In certain embodiments, the hMPV F protein antigen is set forth as:

(SEQ ID NO: 9)
MSWKVVIIFSLLITPQHGLKESYLEESCSTITEGYLSVLRTGWYTNVF
TLEVGDVENLTCSDGPSLIKTELDLTKSALRELKTVSADQLAREEQIE
NPRQSRFVLGAIALGVATAAAVTAGVAIAKTIRLESEVTAIKNALKTT
NEAVSTLGNGVRVLATAVRELKDFVSKNLTRAINKNKCDIpDLKMAVS
FSQFNRRFLNVVRQFSDNAGITPAISLDLMTDAELARAVSNMPTSAGQ
IKLMLENRAMVRRKGFGILIGVYGSSVIYMVQLPIFGVIDTPCWIVKA
APSCSEKKGNYACLLREDQGWYCQNAGSTVYYPNEKDCETRGDHVFCD
TAAGINVAEQSKECNINISTTNYPCKVSTGRHPISMVALSPLGALVAC
YKGVSCSIGSNRVGIIKQLNKGCSYITNQDADTVTIDNTVYQLSKVEG
EQHVIKGRPVSSSFDPIKFPEDQFNVALDQVFENIENSQALVDQSNRI
LSSAEKGNTGFIIVIILIAVLGSSMILVSIFIIIKKTKKPTGAPPELS
GVTNNGFIPHN (A2-D185P).

In certain embodiments, the hMPV F protein antigen is encoded by an mRNA ORF set forth as (SEQ ID NO: 10) (A2-D185P mRNA ORF).

In certain embodiments, the hMPV F protein antigen is encoded by a codon-optimized mRNA ORF set forth as (SEQ ID NO: 17) (AD185P mRNA ORF).

In certain embodiments, the hMPV F protein antigen is set forth as:

(SEQ ID NO: 11)
MSWKVVIIFSLLITPQHGLKESYLEESCSTITEGYLSVLRTGWYTVVF
TLEVGDVENLTCSDGPSLIKTELDLTKSALRELKTVSADQLAREEQIE
NPRQSRFVLGAIALGVATAAAVTAGVAIAKTIRLESEVTAIKNALKTT
NEAVSTLGNGVRVLAFAVRELKDFVSKNLTRAINKNKCDIDDLKMAVS
FSQFNRRFLNVVRQFSDNAGITPAISLDLMTDAELARAVSNMPTSAGQ
IKLMLENRAMVRRKGFGILIGVYGSSVIYMVQLPIFGVIDTPCWIVKA
APSCSEKKGNYACLLREDQGWYCQNAGSTVYYPNEKDCETRGDHVFCD
TAAGINVAEQSKECNINISTTNYPCKVSTGRHPISMVALSPLGALVAC
YKGVSCSIGSNRVGIIKQLNKGCSYITNQDADTVTIDNTVYQLSKVEG
EQHVIKGRPVSSSFDPIKFPEDQFNVALDQVFENIENSQALVDQSNRI
LSSAEKGNTGFIIVIILIAVLGSSMILVSIFIIIKKTKKPTGAPPELS
GVTNNGFIPHN (A2-T160F_N46V).

In certain embodiments, the hMPV F protein antigen is encoded by an mRNA ORF set forth as (SEQ ID NO: 12) (A2-T160F_N46V mRNA ORF).

In certain embodiments, the hMPV F protein antigen is encoded by a codon-optimized mRNA ORF set forth as (SEQ ID NO: 18) (T160F_N46V mRNA ORF).

In certain embodiments, the hMPV F protein antigen is encoded by a codon-optimized mRNA ORF set forth as (SEQ ID NO: 19) (T160F_N46V mRNA ORF).

### II. A. 5′ Cap

An mRNA 5′ cap can provide resistance to nucleases found in most eukaryotic cells and promote translation efficiency. Several types of 5′ caps are known. A 7-methylguanosine cap (also referred to as “m7G” or “Cap-0”) comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5 ′5 ′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; and m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

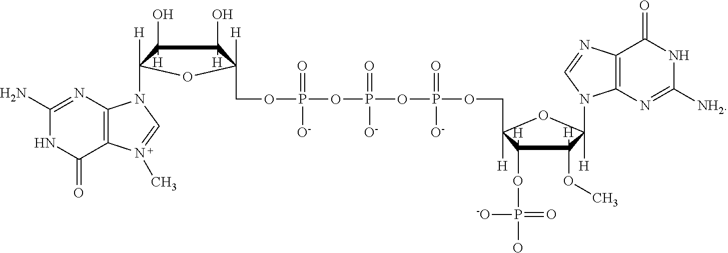

### II. B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 16) (U.S. Publication No. 2016/0151409, incorporated herein by reference in its entirety for all purposes).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 13. In some embodiments, the 3′UTR comprises a nucleic acid sequence set forth in SEQ ID NO: 14. The 5′ UTR and 3′UTR are described in further detail in International Pub. No. WO 2012/075040, incorporated herein by reference.

### II. C. Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence

(SEQ ID NO: 15)
AAAAAAAAAAAAAAAAAAAAAAAAAAAAAAGCAUAUGACUAAAAAAA
AAAAAAAAAAAAAAAAAAAAAAAAAAAAAAAAAAAAAAAAAAAAAAA
AAAAAAAAAAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A)polymerases, e.g., using methods as described in International Pub. No. WO 2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in International Pub. No. WO 2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

### II. D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

### II. E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

### V. Lipid Nanoparticle (LNP)

The LNPs of the disclosure can comprise four categories of lipids: (i) an ionizable lipid (e.g., cationic lipid); (ii) a PEGylated lipid; (iii) a cholesterol-based lipid (e.g., cholesterol), and (iv) a helper lipid.

A. Cationic Lipid

An ionizable lipid facilitates mRNA encapsulation and may be a cationic lipid. A cationic lipid affords a positively charged environment at low pH to facilitate efficient encapsulation of the negatively charged mRNA drug substance. Exemplary cationic lipids are shown below in Table 1.

TABLE 1
Ionizable Lipids

| Name | Structure |
|---|---|
| OF-02 (ML7) | 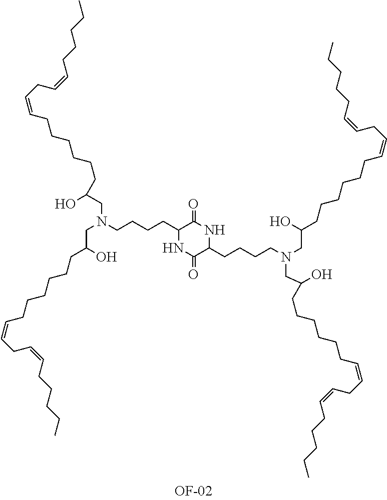 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC] |
| cKK-E10 | 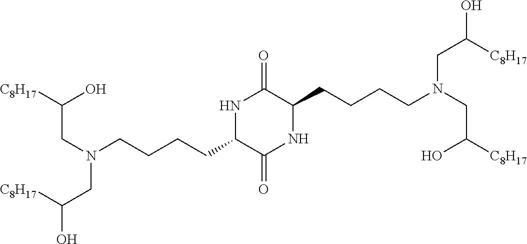 |
| GL-HEPES-E3- E10-DS-3-E18- 1 ((2-(4-(2-((3- (Bis((Z)-2- hydroxyoctadec- 9-en-1- yl)amino)propyl) disulfaneyl)ethyl) piperazin-1- yl)ethyl 4-(bis(2- hydroxydecyl) amino)butanoate) | 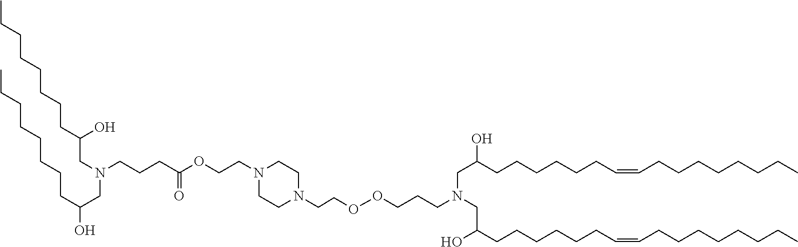 [SMILES: CCCCCCCC/C=C\CCCCCCC(O)CN(CCCOOCCN1CCN(CCOC(=O)CCCN(CC(O)CCCCCCCC)CC(O)CCCCCCCC)CC1)CC(O)CCCCCC/C=C\CCCCCCCC] |
| GL-HEPES-E3- E12-DS-4-E10 (2-(4-(2-((3- (bis(2- hydroxydecyl) amino)butyl)di- sulfaneyl)ethyl) piperazin-1- yl)ethyl 4-(bis(2- hydroxydodecyl) amino)butanoate) | 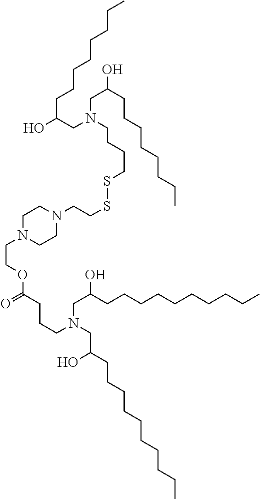 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC] |
| GL-HEPES-E3- E12-DS-3-E14 (2-(4-(2-((3- (Bis(2- hydroxytetra- decyl) amino)propyl) disulfaneyl)ethyl) piperazin-1- yl)ethyl 4-(bis(2- hydroxydodecyl) amino)butanoate) | 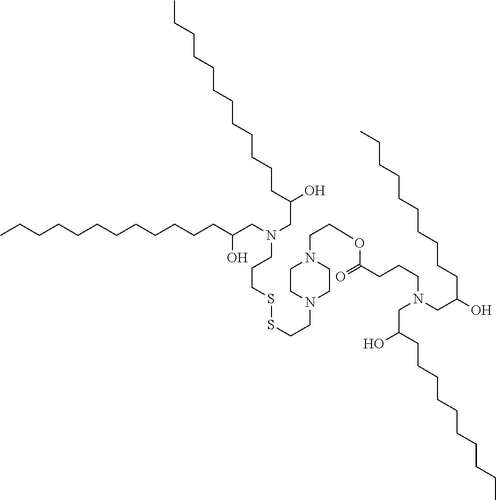 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC] |
| MC3 | 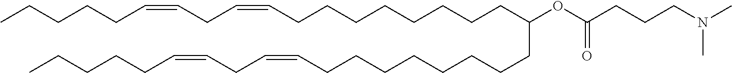 [SMILES: CCCCC/C=C\C/C=C\CCCCCCCCC(CCCCCCCC/C=C\C/C=C\CCCCC)OC(=O)CCCN(C)C] |
| SM-102 (9-heptadecanyl 8-{(2- hydroxyethyl)[6- oxo-6- (undecyloxy) hexyl] amino}octanoate) | 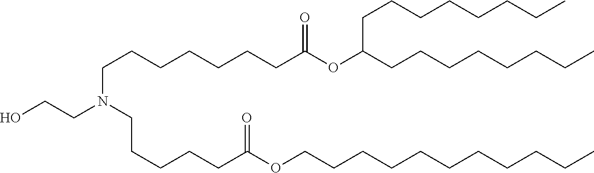 [SMILES: CCCCCCCCCCCOC(=O)CCCCCN(CCO)CCCCCCCC(=O)OC(CCCCCCCC)CCCCCCCC] |
| ALC-0315 [(4-hydroxybutyl) azanediyl]di (hexane-6,1-diyl) bis(2- hexyldecanoate) | 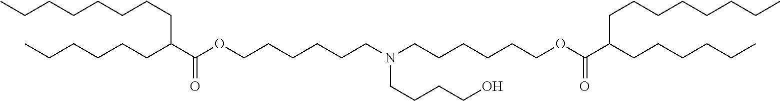 [SMILES: CCCCCCCCC(CCCCCC)C(=O)OCCCCCCN(CCCCO)CCCCCCOC(=O)C(CCCCCC)CCCCCCCC] |

The cationic lipid may be selected from the group comprising [ckkE10]/[OF-02], [(6Z,9Z,28Z,31Z)-heptatriaconta-6,9,28,31-tetraen-19-yl] 4-(dimethylamino)butanoate (D-Lin-MC3-DMA); 2,2-dilinoleyl-4-dimethylaminoethyl[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] N-[2-(dimethylamino)ethyl]carbamate (DC-Chol); tetrakis(8-methylnonyl) 3,3′,3″,3″1-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-016B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9′″,9′″″-((((benzene-1,3,5-tricarbonyl)yris(azanediyl)) tris (propane-3,1-diyl)) tris(azanetriyl)hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); and combinations thereof.

In certain embodiments, the cationic lipid is biodegradable.

In various embodiments, the cationic lipid is not biodegradable.

In some embodiments, the cationic lipid is cleavable.

In certain embodiments, the cationic lipid is not cleavable.

Cationic lipids are described in further detail in Dong et al. (PNAS. 111(11):3955-60. 2014); Fenton et al. (Adv Mater. 28:2939. 2016); U.S. Pat. Nos. 9,512,073; and 10,201,618, each of which is incorporated herein by reference.

B. PEGylated Lipid

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268(1):235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)] (C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

C. Cholesterol-Based Lipid

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), I,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; International Pub. No. WO 2011/068810), sitosterol (22,23-dihydrostigmasterol), β-sitosterol, sitostanol, fucosterol, stigmasterol (stigmasta-5,22-dien-3-ol), ergosterol; desmosterol (3ß-hydroxy-5,24-cholestadiene); lanosterol (8,24-lanostadien-3b-ol); 7-dehydrocholesterol (Δ5,7-cholesterol); di hydrolanosterol (24,25-dihydrolanosterol); zymosterol (5α-cholesta-8,24-dien-3ß-ol); lathosterol (5a-cholest-7-en-3ß-ol); diosgenin ((3β,25R)-spirost-5-en-3-ol); campesterol (campest-5-en-3ß-ol); campestanol (5a-campestan-3b-ol); 24-methylene cholesterol (5,24(28)-cholestadien-24-methylen-3ß-ol); cholesteryl margarate (cholest-5-en-3ß-yl heptadecanoate); cholesteryl oleate; cholesteryl stearate and other modified forms of cholesterol. In some embodiments, the cholesterol-based lipid used in the LNPs is cholesterol.

D. Helper Lipid

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), di palmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, l-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE.

E. Molar Ratios of the Lipid Components

The molar ratios of the above components are important for the LNPs' effectiveness in delivering mRNA. The molar ratio of the cationic lipid, the PEGylated lipid, the cholesterol-based lipid, and the helper lipid is A: B: C: D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:
a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);
a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);
a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and
a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),
wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 ata molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: GL-HEPES-E3-E12-DS-4-E10 (at a molar ratio of 40%; DMG-PEG2000 at a molar ratio of 1.5%; cholesterol at a molar ratio of 28.5%; and DOPE at a molar ratio of 30%.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: 9-heptadecanyl 8-{(2-hydroxyethyl)[6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

F. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitation, sucrose, trehalose, glucose, mannitol, mannose, dextrose, and the like. The cryoprotectant may constitute 5-30% (w/v) of the LNP composition. In some embodiments, the LNP composition comprise trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution—thawed if previously frozen, or if previously lyophilized, reconstituted in an aqueous buffered solution at bedside. The buffered solution can be isotonic and suitable, e.g., for intramuscular or intradermal injection. In some embodiments, the buffered solution is a phosphate-buffered saline (PBS).

### VI. Processes for Making LNP Vaccines

The present LNPs can be prepared by various techniques. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication, or extrusion of the multilamellar vesicles. In addition, unilamellar vesicles can be formed by detergent removal techniques.

Various methods are described in U.S. Publication Nos. US 2011/0244026, US 2016/0038432, US 2018/0153822, US 2018/0125989, and US 2021/0046192 and can be used for making LNP vaccines. One exemplary process entails encapsulating mRNA by mixing it with a mixture of lipids, without first pre-forming the lipids into lipid nanoparticles, as described in U.S. Publication No. US 2016/0038432. Another exemplary process entails encapsulating mRNA by mixing pre-formed LNPs with mRNA, as described in U.S. Publication No. US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA, and the mixed solution comprising the LNP-encapsulated mRNA. In some embodiments, the process includes the step of heating one or both of the mRNA solution and the pre-formed LNP solution prior to the mixing step. In some embodiments, the process includes heating one or more of the solutions comprising the pre-formed LNPs, the solution comprising the mRNA, and the solution comprising the LNP-encapsulated mRNA during the mixing step. In some embodiments, the process includes the step of heating the LNP-encapsulated mRNA after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present disclosure. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include, but are not limited to, gear pumps, peristaltic pumps, and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging from about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging from about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods are available for sizing of a population of lipid nanoparticles. In various embodiments, methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80% or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In certain embodiments, the LNP has an average diameter of 30-200 nm.

In various embodiments, the LNP has an average diameter of 80-150 nm.

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, or 99% of the LNPs in the present composition have a size ranging from about 40-90 nm (e.g., about 45-85 nm, about 50-80 nm, about 55-75 nm, or about 60-70 nm) or about 50-70 nm (e.g., about 55-65 nm) are suitable for pulmonary delivery via nebulization.

In some embodiments, the dispersity, or measure of heterogeneity in size of molecules (PDI), of LNPs in a pharmaceutical composition provided by the present disclosure is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of 50% to 99%; or greater than about 60%, 65%, 70%, 75%, 80%, 85%, 90%, 92%, 95%, 98%, or 99%. Typically, lipid nanoparticles for use herein have an encapsulation efficiency of at least 90% (e.g., at least 91%, 92%, 93%, 94%, or 95%).

In some embodiments, an LNP has a N/P ratio of 1 to 10. In some embodiments, a lipid nanoparticle has a N/P ratio above 1, about 1, about 2, about 3, about 4, about 5, about 6, about 7, or about 8. In certain embodiments, a typical LNP herein has an N/P ratio of 4.

In some embodiments, a pharmaceutical composition according to the present disclosure contains at least about 0.5 μg, 1 μg, 5 μg, 10 μg, 100 μg, 500 μg, or 1000 μg of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. In this process, an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 4.5.

An exemplary, nonlimiting process for making an mRNA-LNP composition involves mixing a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

### VII. Packaging and Use of the hMPV Vaccine

One or more hMPV F polypeptide antigens described herein may be administered to a subject as a vaccine. hMPV vaccines described herein can be formulated or packaged for parenteral (e.g., intramuscular, intradermal, or subcutaneous) administration or nasopharyngeal (e.g., intranasal) administration. In various embodiments, the hMPV vaccines may be formulated or packaged for pulmonary administration. In various embodiments, the hMPV vaccines may be formulated or packaged for intravenous administration. The vaccine compositions may be in the form of an extemporaneous formulation, where the composition is lyophilized and reconstituted with a physiological buffer (e.g., PBS) just before use. The vaccine compositions also may be shipped and provided in the form of an aqueous solution or a frozen aqueous solution and can be directly administered to subjects without reconstitution (after thawing, if previously frozen).

Accordingly, the present disclosure provides an article of manufacture, such as a kit, that provides the hMPV vaccine in a single container or provides the hMPV vaccine in one container (e.g., a first container) and a physiological buffer for reconstitution in another container (e.g., a second container). The container(s) may contain a single-use dosage or multi-use dosage. The container(s) may be pre-treated glass vials or ampules. The article of manufacture may include instructions for use as well.

Methods of administration of an hMPV vaccine include, but are not limited to, intradermal, intramuscular, intraperitoneal, intravenous, subcutaneous, intranasal, intra-tracheal, epidural, and oral routes. The composition may be administered by any convenient route, for example, by infusion or bolus injection, by absorption through epithelial or mucocutaneous linings (e.g., oral mucosa, rectal and intestinal mucosa, etc.) and may be administered together with other biologically active agents.

In particularly exemplary embodiments, a vaccine is administered intramuscularly (IM) by injection. The hMPV vaccine can be injected into a subject at, e.g., their deltoid muscle in the upper arm. In such embodiments, injectables are prepared in conventional forms, i.e., either as liquid solutions or suspensions, solid forms suitable for solution or suspension in liquid prior to injection, or as emulsions. In some embodiments, injection solutions and suspensions are prepared from sterile powders, lyophilized powders, or granules.

A pharmaceutical composition described herein can be delivered, e.g., intramuscularly, subcutaneously, or intravenously, with a standard needle and syringe, which is optionally prefilled. In addition, a pen delivery device (e.g., an injector (e.g., single-chambered or multi-chambered) or an autoinjector pen) has applications in delivering a pharmaceutical composition described herein. Such a pen delivery device can be reusable or disposable. In some embodiments, the vaccine is provided for use in inhalation and is provided in a pre-filled pump, aerosolizer, or inhaler. In certain embodiments, a prefilled syringe may be utilized for drop-wise administration for intranasal delivery.

The hMPV vaccines can be administered to subjects in need thereof in a prophylactically effective amount, i.e., an amount that provides sufficient immune protection against a target pathogen for a sufficient amount of time (e.g., one year, two years, five years, ten years, or a lifetime). Sufficient immune protection may be, for example, prevention or alleviation of symptoms associated with infections by the pathogen. In some embodiments, multiple doses (e.g., two doses) of the vaccine are administered (e.g., injected) to subjects in need thereof to achieve the desired prophylactic effects. The doses (e.g., prime and booster doses) may be separated by an interval of at least, e.g., 2 weeks, 3 weeks, 4 weeks, one month, two months, three months, four months, five months, six months, one year, two years, five years, or ten years.

### VIII. Pharmaceutical Compositions

hMPV polypeptide antigens purified according to this disclosure can be useful as a component in pharmaceutical compositions, for example, for use as a vaccine. These compositions will typically include RNA or a binding polypeptide and a pharmaceutically acceptable carrier. A pharmaceutical composition of the present disclosure can also include one or more additional components such as small molecule immunopotentiators (e.g., TLR agonists). A pharmaceutical composition of the present disclosure can also include a delivery system for the RNA, such as a liposome, an oil-in-water emulsion, or a microparticle. In some embodiments, the pharmaceutical composition comprises a lipid nanoparticle (LNP). In certain embodiments, the composition comprises an antigen-encoding nucleic acid molecule encapsulated within an LNP.

Methods that comprise administering an hMPV binding polypeptide to a patient, wherein the hMPV binding polypeptide antagonist is contained within a pharmaceutical composition are provided. The pharmaceutical compositions described herein are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol. 52:238-311.

Various delivery systems are known and can be used to administer the pharmaceutical compositions described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition described herein can be delivered subcutaneously or intravenously with a standard needle and syringe (e.g., a prefilled syringe). In addition, with respect to subcutaneous delivery, a pen delivery device (e.g., an autoinjector pen) readily has applications in delivering a pharmaceutical composition described herein.

For direct administration to the sinuses, the pharmaceutical compositions described herein may be administered using, e.g., a microcatheter (e.g., an endoscope and microcatheter), an aerosolizer, a powder dispenser, a nebulizer, or an inhaler. The methods include administration of an hMPV binding polypeptide to a subject in need thereof in an aerosolized formulation. Aerosolized antibodies can be prepared as described in, for example, U.S. Pat. No. 8,178,098, incorporated herein by reference in its entirety.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending, or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### IX. Methods of Vaccination

The hMPV vaccine disclosed herein may be administered to a subject to induce an immune response directed against the hMPV F protein, wherein an anti-antigen antibody titer in the subject is increased following vaccination relative to an anti-antigen antibody titer in a subject that is not vaccinated with the hMPV vaccine disclosed herein, or relative to an alternative vaccine against hMPV. An “anti-antigen antibody” is a serum antibody that binds specifically to the antigen.

In one aspect, the disclosure provides a method of eliciting an immune response to hMPV or protecting a subject against hMPV infection comprising administering an hMPV vaccine described herein to a subject. The disclosure also provides an hMPV vaccine described herein for use in eliciting an immune response to hMPV or in protecting a subject against hMPV infection. The disclosure also provides an hMPV mRNA described herein for use in the manufacture of a vaccine for eliciting an immune response to hMPV or for protecting a subject against hMPV infection.

In certain embodiments, the subject has a comparable serum concentration of neutralizing antibodies against hMPV after administration of the hMPV vaccine, relative to a subject that is administered an hMPV protein vaccine that is co-administered with an adjuvant.

In certain embodiments, the hMPV vaccine increases the serum concentration of antibodies with binding specificity to site 0 of the hMPV F protein.

In certain embodiments, the hMPV vaccine increases the serum concentration of antibodies with binding specificity to site V of the hMPV F protein.

In certain embodiments, the hMPV vaccine increases the serum concentration of neutralizing antibodies in a subject with pre-existing hMPV immunity.

In order that this invention may be better understood, the following examples are set forth. These examples are for purposes of illustration only and are not to be construed as limiting the scope of the invention in any manner.

### EXAMPLES

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 1: Generation of the Pre-Fusion Stabilized hMPV F Glycoprotein Antigen Constructs

To improve the stability of the prefusion conformation, enhance purification, and induce higher neutralizing antibody titers, a panel of candidate hMPV prefusion F antigen constructs were designed with mutations in the wild-type hMPV-F antigen based on the A2 subtype from Canada designated A2-CAN97-83 (SEQ ID NO: 1).

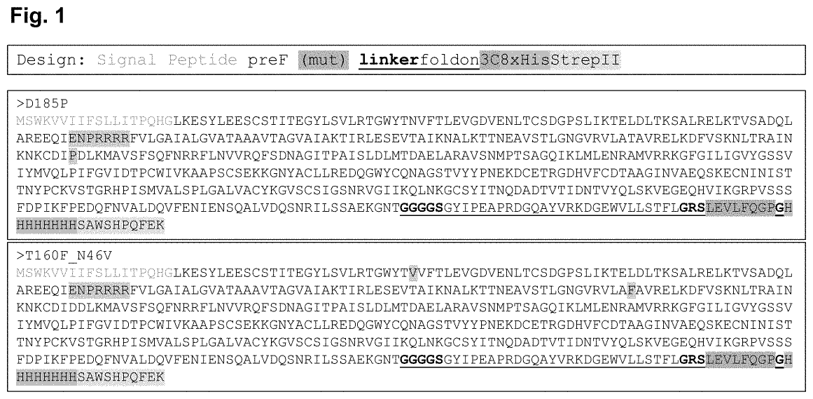

A graphical representation of the design considerations for the panel of candidate hMPV prefusion F antigen constructs are shown in FIG. 1 for two exemplary constructs, D185P (SEQ ID NO: 5) and T160F/N46V (SEQ ID NO: 7). Each construct contained the following characteristics: (1) signal peptide; (2) pre-F cleavage site mutations at amino acid 100-101 (QS to RR); (3) removal of transmembrane domain and cytoplasmic tail; (4) addition of a fibritin motif (i.e., a foldon domain); (5) HRV-3C cleavage site; (6) 8×His tag and Strep II tags; and (7) appropriate linkers for items (4) through (6) (SEQ ID NO: 3).

From this backbone, an in silico analysis was performed to determine single- or double-point mutations that would increase pre-F conformation stability by adding either filling cavity mutations or interface stability mutations. In total, the panel of candidate hMPV prefusion F antigens was comprised of 21 different constructs as shown in column 1 of Table 1.

### Example 2: Evaluation of Protein Expression for the Pre-Fusion Stabilized hMPV F Antigen Constructs

The nucleic acid molecule for each of the candidate hMPV prefusion F antigen constructs was isolated and cloned into an expression vector. Production of protein expression for each construct was evaluated upon mammalian transient transfection using Expi293F human cells. Twenty-four hours after transfection of the constructs, cell lysates or supernatants were recovered for analysis by western blot.

Out of the 21 candidate designs, nine protein antigens were produced. However, only four protein antigens had ≥90% purity as determined by SDS-PAGE from a 1 L culture. Protein expression characteristics for all of the 21 constructs are shown in Table 1. Constructs that had high protein production and purity had the following mutations: D185P, T160F_N46V, K138F, and G366F_K362F.

TABLE 1
Protein expression characteristics of the 21-
candidate hMPV prefusion F antigen constructs

| ID # | Single or Double Mutations | 1st culture | Purity by SDS-PAGE |
|---|---|---|---|
| 1 | D185P | High | >95% |
| 2 | G366F_K362Y | N/D | |
| 3 | K126F_E431A | N/D | |
| 4 | D87F_E327F | N/D | |
| 5 | R253F_E327F | N/D | |
| 6 | Y425F_A116Y | Low | >70% |
| 7 | Q426F_T119F | Low | >75% |
| 8 | T160F_N46V | High | >95% |
| 9 | K166F_E51F | N/D | |
| 10 | E327F_R329F | N/D | |
| 11 | R304K_D306W | N/D | |
| 12 | T160F_T49Y | N/D | |
| 13 | K126A | N/D | |
| 14 | K138F | High | >90% |
| 15 | T83F_K75F | N/D | |
| 16 | K362L_D454E | Low | >75% |
| 17 | E305F | N/D | |
| 18 | A159F | Low | >60% |
| 19 | G366F_K362F | High | >95% |
| 20 | G366D | Low | >40% |
| 21 | K126F_E431F | N/D | |

### Example 3: Immunogenicity of the Pre-Fusion Stabilized hMPV F Antigen Protein Constructs in Mice

The four candidate hMPV F antigen constructs with 90% purity described in Table 1 were subsequently evaluated for immunogenicity in mice as compared to reference hMPV-F protein from an A1 strain.

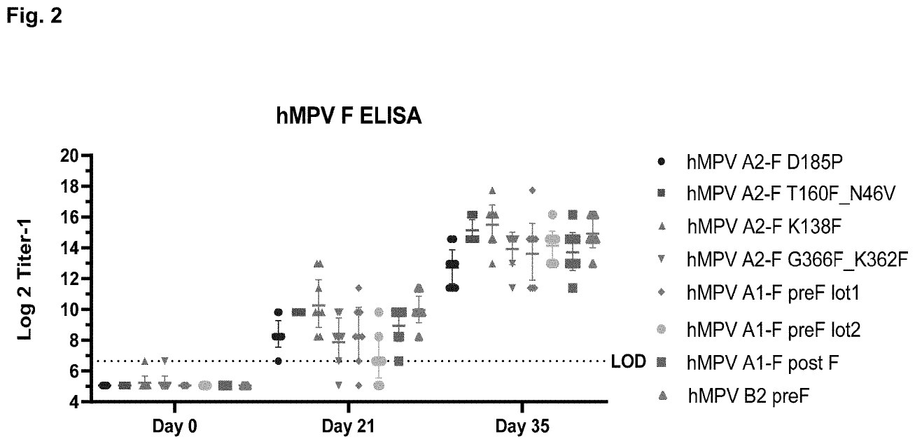

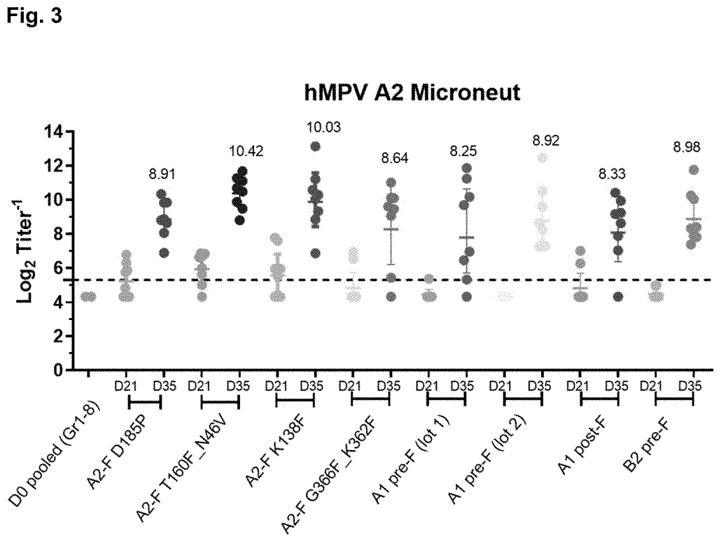

Groups of 8 BALB/c mice (N=8) as shown in Table 2 were administered a 0.5 μg dose of protein antigen adjuvanted with aluminum hydroxide (Al(OH)₃) by intramuscular (IM) injection on day (D) 0 and D21. All mice were bled and sera was extracted prior to each vaccine administration as well as at two weeks post-last vaccination (D35). Sera was then used to determine the circulating anti-hMPV-F IgG titers as measured by enzyme-linked immunosorbent assay (ELISA) (FIG. 2) and by hMPV microneutralization assay (FIG. 3) to determine the neutralizing activity of the antibody responses. To ensure that all proteins in the post-F group were indeed in the post-F conformation, the proteins were heated to 70° C. for 10 minutes prior to preparation for administration.

TABLE 2
hMPV F antigen vaccine study design in mice

| | No. | Protein antigen | | | |
|---|---|---|---|---|---|
| | of | plus adjuvant | Dose | | |
| Group | mice | (alum) | (μg) | Administration | Rationale |
| 1 | 8 | hMPV A2-F D185P | 0.5 | 50 μl per leg | Test |
| | | | | (100 μl total) | Construct |
| 2 | 8 | hMPV A2-F | 0.5 | 50 μl per leg | Test |
| | | T160F_N46V | | (100 μl total) | Construct |
| 3 | 8 | hMPV A2-F K138F | 0.5 | 50 μl per leg | Test |
| | | | | (100 μl total) | Construct |
| 4 | 8 | hMPV A2-F | 0.5 | 50 μl per leg | Test |
| | | G366F_K362F | | (100 μl total) | construct |
| 5 | 8 | hMPV A1 pre-F | 0.5 | 50 μl per leg | A1 pre-F |
| | | lot 1 | | (100 μl total) | reference |
| 6 | 8 | hMPV A1 pre-F | 0.5 | 50 μl per leg | A1 pre-F |
| | | lot 2 | | (100 μl total) | reference |
| 7 | 8 | hMPV A1 post-F | 0.5 | 50 μl per leg | A1 post-F |
| | | (heat-treated) | | (100 μl total) | reference |
| 8 | 8 | hMPV B2 pre-F | 0.5 | 50 μl per leg | B2 pre-F |
| | | | | (100 μl total) | reference |

The data shows that the construct with the A2-K138F mutation induced the highest binding antibody titer by hMPV-F ELISA followed by A2-T160F_N46V, A2-G366F_K362F, and finally A2-D185P (FIG. 2). When evaluated by microneutralization using an hMPV A2-GFP virus, A2-T160F_N46V had the highest neutralization titer followed by A2-K138F, A2-D185P, and A2-G366F_K362F (FIG. 3).

Although A2-K138F had the highest binding antibody titer and second highest neutralizing antibody titer, this construct was found to form aggregates in solution, indicating potential improper protein folding, and was thus eliminated from further evaluation. A2-G366F_K362F was also eliminated from further evaluation as it had the second lowest binding antibody titer and the lowest neutralizing antibody titer. Therefore, A2-D185P and A2-T160F_N46V were found to induce the highest quality antibodies, and were chosen for advanced analytic analysis to evaluate purity, size and thermal stability as described in Example 4.

### Example 4: Physicochemical Characterization of the Pre-Fusion Stabilized hMPV F Antigen Constructs

To further characterize the purity, size, and thermal stability of the protein produced from the A2-D185P and A2-T160F_N46V constructs—HP-SEC, SEC-HPLC, SEC-MALS, and nanoDSF analysis was performed.

Purity and Size

The results for the HP-SEC, SEC-HPLC, and SEC-MALS analysis are summarized below in Table 3.

TABLE 3
Summary of SEC evaluations for the pre-fusion stabilized
hMPV F antigen constructs and controls.

| | A1 | A1 | A2 | A2 |
|---|---|---|---|---|
| | A185P | Post-F | T160F_N46V | D185P |
| HP-SEC Trimer (%) | 98.8 | 100 | 94.7 | 97.1 |
| SEC-HPLC MW (kDa) | 341.4 | 337.2 | 384.0 | 321.7 |
| SEC-MALS MW (KDa) | 224.4 | 282.5 | 266.5 | 224.3 |

Molecular weight (MW) from MALS was determined for trimer peak. Conditions for SEC-HPLC were as follows: TSK 3000SWxl SEC column, Phosphate Buffer (0.2M NaH₂PO₄, 0.1M Arginine, 1% IPA, pH 6.5), flow rate 0.5 ml/min. Conditions for SEC-MALS were as follows: 1.7 μM, 200 Å BEH Protein Column, and 50 mM Tris buffer at pH 7.5, flow rate 0.3 ml/min.

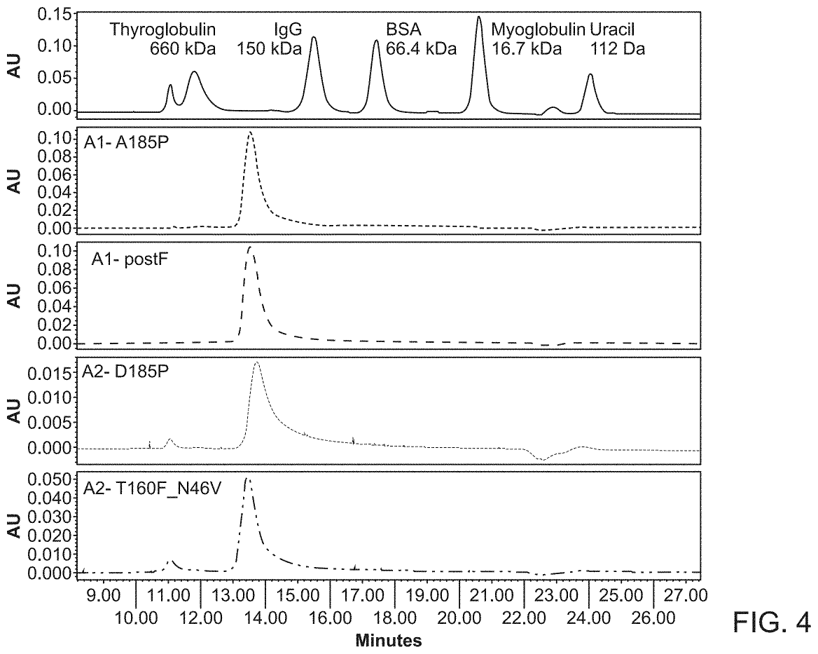

FIG. 4 displays the SEC-MALS results for the reference A1 proteins, A1-A185P and A1-post-F, and below, the A2 protein antigen candidates, A2-T160F_N46V and A2-D185P. Data for all four proteins is also summarized in Table 3. Both A1 reference proteins show 98.8% trimer formation and a MW of 224 and 283 kDa for A1-A185P and A1-post-F, respectively. Protein from the A2-T160F_N46V and A2-D185P constructs was composed of 97.4% and 97.1% trimer with a MW of 267 and 224 kDa, respectively.

Thermal Stability

Onset temperatures (Tonset) and melting points (Tm) of protein denaturation were determined using nanoscale differential scanning fluorimetry (nanoDSF) on both large and small batch lots of A1-pre-F and A1-post-F proteins as well as the A2 candidate protein antigens, A2-T160F_N46V and A2-D185P. Samples were diluted in formulation buffer to a final concentration of 0.5 mg/ml and loaded into nanoDSF capillaries in duplicates. All measurements were done using a nanoDSF device. Heating rate was 1.5° C. per minute from 20° C. to 95° C. Data were recorded and analyzed using PR.Stability Analysis v1.01.

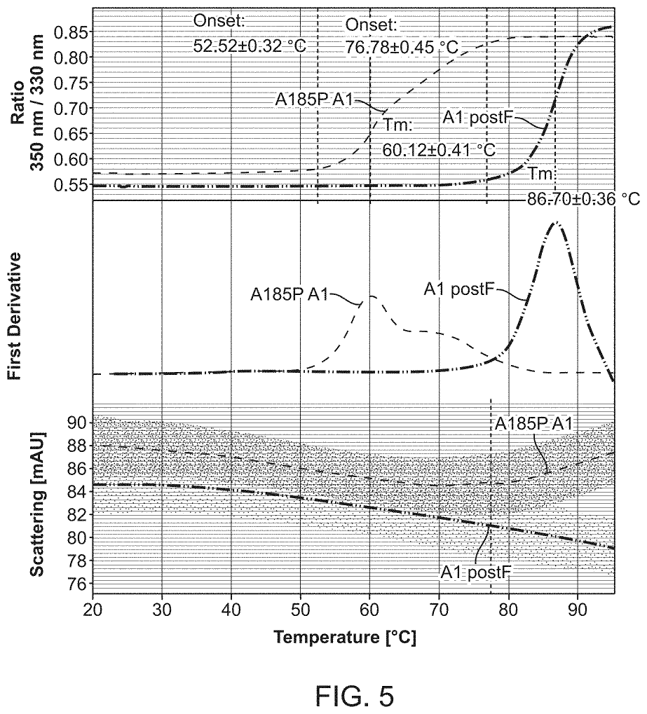

FIG. 5 shows melting curves for A1-preF (A185P) and A2-postF (n=3), yielding Tm values of 60.12° C. and 86.7° C., respectively. This data shows that nanoDSF could differentiate between A1 pre- and post-fusion antigens, with approximately a 27° C. difference in the melting temperature.

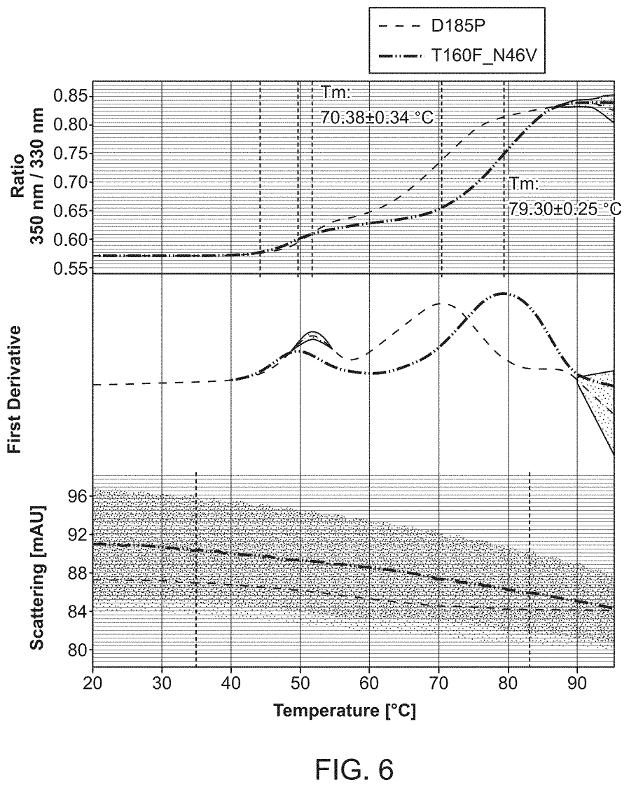

Interestingly, when comparing the thermostability properties of the A2 hMPV-F candidate protein antigens, as seen in FIG. 6, protein derived from the A2-T160F_N46V construct was found to be more thermostable than the more minimally engineered protein produced from the A2-D185P construct, with a melting point increase of nearly 9° C. (Tm 70.4° C. and 79.3° C., respectively).

### Example 5: mRNA Encoding Pre-Fusion Stabilized hMPV F Antigen Constructs

To determine whether immunogenicity of the hMPV F antigen constructs could be further improved, the A2-D185P and A2-T160F_N46V constructs were selected for testing in an mRNA-based vaccine. The amino acid sequences for the A2-D185P and A2-T160F_N46V constructs are set forth in SEQ ID NO: 9 and SEQ ID NO: 11, respectively. The mRNA ORFs for the A2-D185P and A2-T160F_N46V constructs are set forth in SEQ ID NO: 10 and SEQ ID NO: 12, respectively. Codon-optimized mRNA ORFs for the A2-D185P and A2-T160F_N46V constructs are set forth in SEQ ID NO: 17 and SEQ ID NO: 18, respectively.

The mRNAs described herein comprised an open reading frame (ORF) encoding an hMPV F protein antigen, at least one 5′ untranslated region (5′ UTR), at least one 3′ untranslated region (3′ UTR), and at least one polyadenylation (poly(A)) sequence. The mRNAs further comprised a 5′ cap with the following structure:

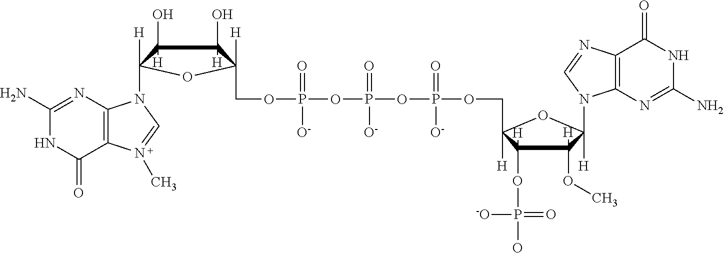

The nucleic acid sequences for the 5′ UTR and 3′ UTR are recited in SEQ ID NO: 13 and 14, respectively.

### Example 6: Immunogenicity of the Pre-Fusion Stabilized hMPV F Antigen mRNA Constructs in Mice

The relative immunogenicity of the A2-D185P and A2-T160F_N46V constructs expressing mRNA was tested in mice by measuring the circulating anti-hMPV-F titers before and after IM injection with mRNA formulated with a Lipid Nanoparticle (LNP). Each mRNA was encapsulated into an LNP composed of 40% cationic lipid OF-02, 30% phospholipid DOPE, 1.5% PEGylated lipid DMGPEG2000, and 28.5% cholesterol. Alternatively, the LNP lipids may be recited as ratios where cationic lipid:PEGylated lipid:cholesterol:phospholipid is 40:1.5:28.5:30.

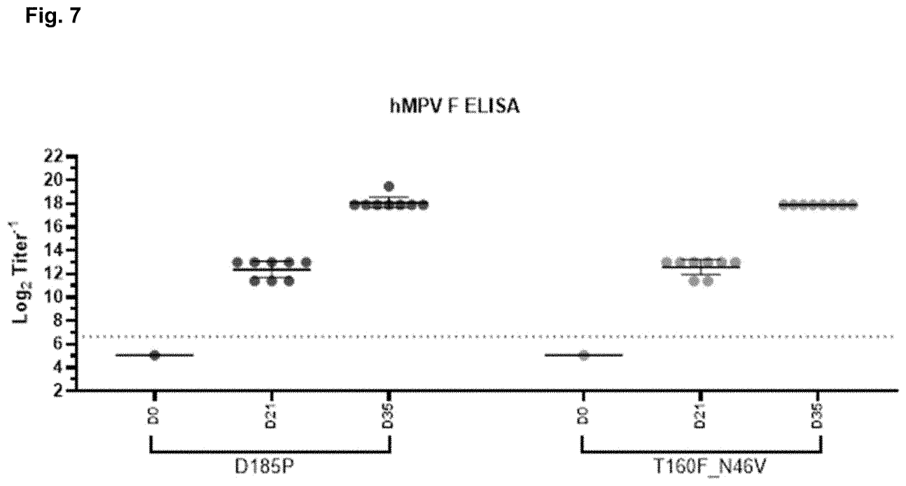

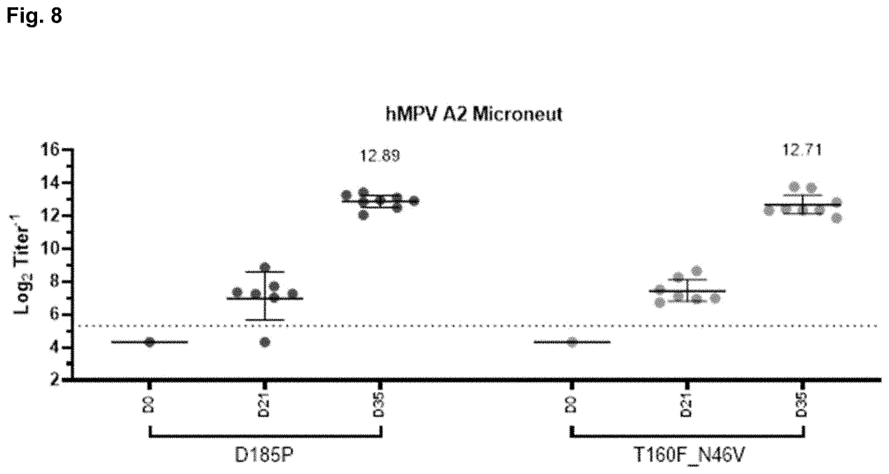

Groups of eight BALB/c mice (N=8) were administered a 1 μg dose via IM injection on DO and D21. All mice were bled and sera was extracted prior to each vaccine administration, as well as at two weeks post-last vaccination (D35). Sera was then used to determine the circulating anti-hMPV-F IgG titers as measured by ELISA (FIG. 7) and by hMPV microneutralization assay (FIG. 8) to determine the neutralizing activity of the antibody responses.

Both A2-D185P and A2-T160F_N46V constructs expressing mRNA induced similarly high titers of binding antibodies at all timepoints by hMPV-F ELISA (FIG. 7). When evaluated by microneutralization using an A2-GFP virus, similarly potent neutralization titers were induced by both constructs (FIG. 8). Thus, both antigens were similarly immunogenic when expressed via mRNA-LNP.

### Example 7: Rational Design of an mRNA Multi-Pathogen Vaccine Directed to hMPV and RSV

RSV and hMPV are respiratory viruses that cause widespread morbidity within the human population second only to influenza virus (Collins et al. 2013, Fields Virology. 6 ed: Lippincott Williams and Wilkins). Despite the disease burden, vaccine and therapeutic strategies for both viruses remain limited. Given the substantial homology between the hMPV and RSV surface glycoproteins, as well as the practical consideration that protection against both viruses would result in fewer injections and simplify vaccine schedules (Lauer et al. 2017, Clin Vaccine Immunol. 24(1):e00298-16), a combination mRNA vaccine comprising the RSV and hMPV antigen constructs was designed.

Accordingly, a combination vaccine comprising two mRNAs: (1) RSV F antigen construct, FD3; and (2) hMPV F antigen construct, A2-CAN97-83 was co-formulated.

The mRNA hMPV construct, as well as the 5′ and 3′ UTRs used are described in Example 6.

The mRNA RSV construct used is described in U.S. Provisional Patent Application Ser. No. 63/276,233, which is incorporated herein by reference in its entirety for all purposes.

### Example 8: Immunogenicity of the mRNA Multi-Pathogen Vaccine Directed to hMPV and RSV in Mice

The relative immunogenicity of the mRNA multi-pathogen vaccine directed to hMPV and RSV as described in Example 7 was evaluated in mice by measuring the circulating anti-RSV FD3 and anti-hMPV-F titers before and after IM injection with mRNA formulated with a lipid nanoparticle (LNP). Each mRNA was encapsulated into an LNP composed of 40% cationic lipid OF-02, 30% phospholipid DOPE, 1.5% PEGylated lipid DMGPEG2000, and 28.5% cholesterol. Alternatively, the LNP lipids may be recited as ratios where cationic lipid:PEGylated lipid:cholesterol:phospholipid is 40:1.5:28.5:30.

Five groups of BALB/c mice (N=8) were immunized via IM injection on DO and D21 according to the following regimens: (1) Group 1—were administered a 1 μg dose of RSV mRNA formulated with cOrn-EE1 LNP in 50 μL total volume administered to the right hind limb; (2) Group 2—were administered 1 μg dose of hMPV mRNA formulated with cOrn-EE1 LNP in 50 μL total volume administered to the right hind limb; (3) Group 3—were administered a co-formulation containing 1 μg of RSV and 1 μg of hMPV mRNA in a single cOrn-EE1 LNP in a total volume of 100 μL, with 50 μL delivered to each hind limb; (4) Group 4—were administered a 1 μg of RSV pre-F protein nanoparticle adjuvanted with Alum in 50 μL total volume administered to the right hind limb as RSV immunogenicity control; and (5) Group 5—were administered 1 μg of hMPV pre-F adjuvanted with Alum in 50 μL total volume administered to the right hind limb as hMPV immunogenicity control.

All mice were bled prior to each vaccine administration as well as at 2 weeks post-last vaccination (D35) and the D35 sera were tested by ELISA to determine circulating anti-RSV and anti-hMPV-F titers and by microneutralization assays to determine the neutralizing activity of the RSV and hMPV antibody responses.

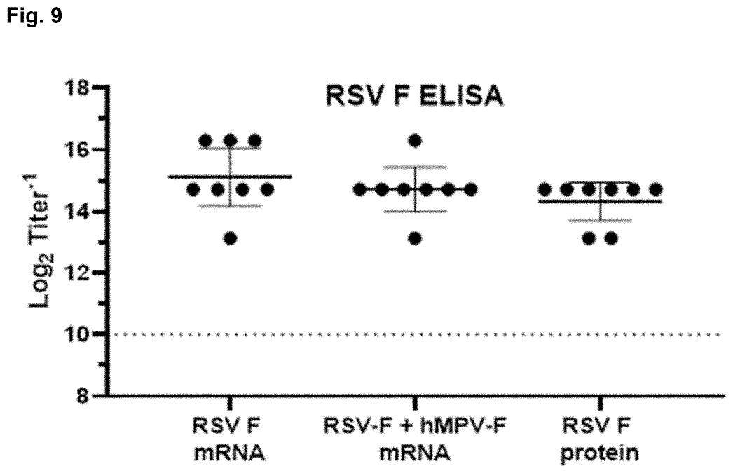

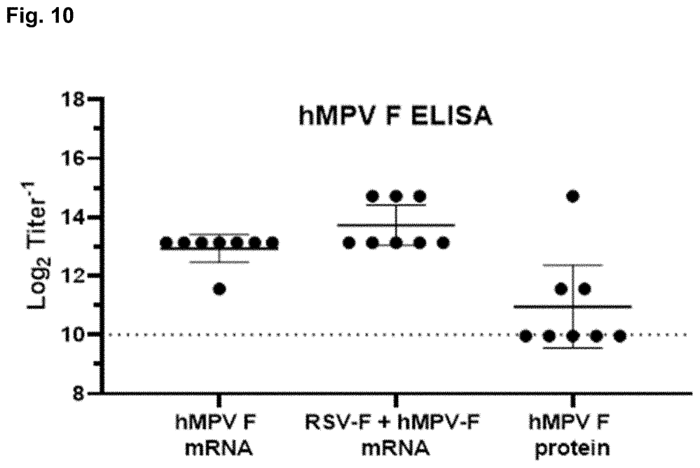

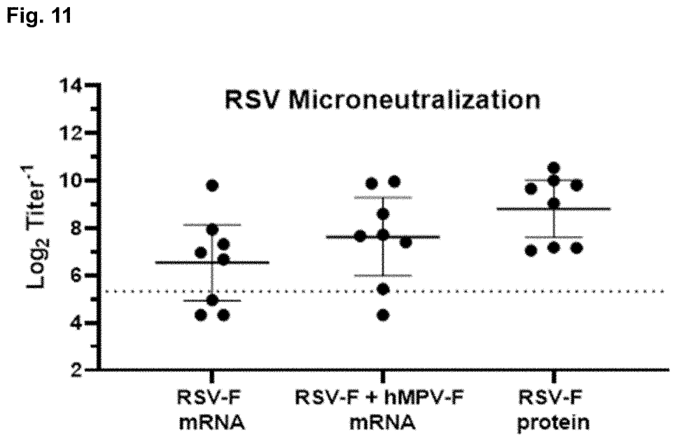

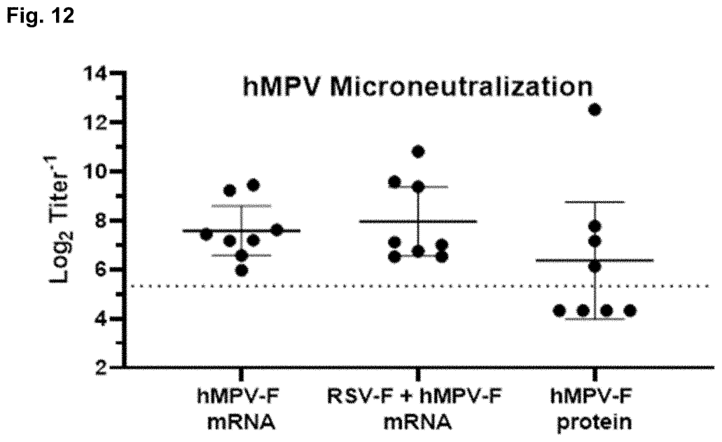

RSV mRNA induced similar titers by RSV-F ELISA when given alone or when co-formulated with hMPV (FIG. 9). Similarly, hMPV mRNA induced similar titers by hMPV-F ELISA when given alone or when co-formulated with RSV (FIG. 10). When evaluated by microneutralization using an RSV A2-GFP virus, similarly potent neutralization titers were induced by RSV mRNA delivered alone or in combination with hMPV (FIG. 11). When evaluated by microneutralization using an hMPV A2-GFP virus, similarly potent neutralization titers were induced by hMPV mRNA delivered alone or in combination with RSV (FIG. 12). Thus, immunization with a co-formulation of RSV and hMPV mRNA delivered as a single LNP is similarly immunogenic to either antigen delivered alone.

### Example 9: Analysis of hMPV F Polypeptide Antigen-Antibody Binding

Binding of antibodies to hMPV F constructs was tested in octet. All samples and antibodies were diluted in kinetic buffer (ForteBio Kinetic buffer 1× dilution+PBS) to final concentrations of 5 μg/mL and 1 μg/mL, respectively. Antibodies were loaded onto Protein A biosensors and the binding of all antigens was tested with the following conditions: initial baseline (120 s), loading of the antibody (180 s), second baseline (120 s), association of the antigen (180 s), dissociation of the antigen (120 s). Binding results were analyzed using ForteBio Data Analysis 12.0 software.

Table 4 shows that A2-T160F_N46V and A2-D185P had expected binding patterns for the mAbs MPEG, 101F, 338, and DS7.

TABLE 4
Binding characteristics of hMPV F antigenic constructs

| | | A1 pre-F | | A2 pre-F | A2 pre-F |
|---|---|---|---|---|---|
| Antibody | Site | (A185P) | A1 post-F* | (D185P) | (T160F/N46V) |
| MPE8 | III | + | − | + | + |
| 101F | IV | + | + | + | + |
| 338 | II | + | + | + | + |
| DS7 | I | + (low) | + | + (low) | + (low) |

### Example 10: hMPV F Antigen Expression

### HEK293 Cells—0.3 Million Cells/Well

Next, HELA cells were plated in 6-well plates at 0.3 million cells/well in 2 mL DMEM+10% FBS. Cells were transfected the next day with 1 μg/well hMPV mRNA constructs with lipofectamine 2000. Cells were harvested the next day and lysed in 500 μL per well of RIPA+1× HALT+0.2% Omnicleave. Lysates were incubated on ice for 10 minutes. 15 μL Lysate was combined with 5 μL NuPAGE LDS Sample buffer.

Samples were run on 8-16% Gradient SDS-PAGE at 185 V for 75 minutes. Protein was transferred to nitrocellulose membrane. Blots were blocked with Intercept protein free blocking buffer for 1 hour at room temperature. Blots were stained with primary antibody, NBP2-50505 Mouse Anti-hMPV-F (hMPV24) (Novus), in Intercept protein free Blocking buffer overnight at 4° C. Blots were washed with TBST 3×5 minutes. Blots were stained with donkey anti-mouse 800 secondary antibody in Intercept Blocking buffer for 1 hour at room temperature. Blots were washed with TBST 4×5 minutes, and then scanned on Licor Odyssey.

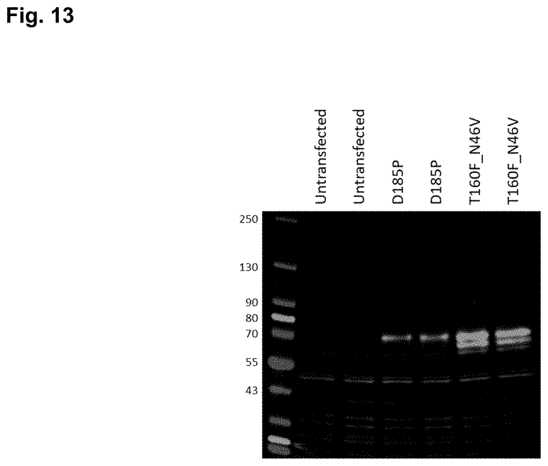

Results are shown at FIG. 13. D185P showed robust expression near the expected molecular weight of 60 kDa. T160F_N46V showed protein expression but the signal was significantly lower than D185P.

### HSKM Cells

mRNAs were transfected into human skeletal muscle (HSKM) cells in 24-well plates. After 24 hours, flow cytometry was used to measure epitope expression. Number of cells/well=100k. After 24 hours of transfection, wells were washed with PBS, and trypsin (0.25%) was added to detach the cells. Cells were distributed in 96-well plates for flow cytometry and treated for intracellular staining (Cytoperm) before adding the antibodies. Secondary antibody was Goat anti-Human IgG (Jackson Immuno Research—Cat. #109-115-098).

MNR hMPV D185P expression levels were similar to MNR hMPV CAN97-83. MNR hMPV T160F_N46V showed higher expression levels for all epitopes.

### Example 11: Immunogenicity of Pre- and Post-Stabilized hMPV F Antigen Protein Constructs in a MIMIC System

### Introduction

The MIMIC© (Modular Immune In vitro Construct) system can stimulate innate and adaptive immune responses in vitro that occur in vaccination/inflection site in vivo. Williams et al. (2015) Sanofi Pasteur poster, “In vitro differentiation of class-switched YF specific antibody secreting cells from naïve B cells.” Using the MIMIC system can recapitulate some aspects unique to human physiology, e.g., HLA haplotypes, age, autoimmune statue, and gender, thereby complementing immunogenicity studies performed in animal models. Higbee et al. (2009) ATLA 37: 19-27.

To this end, pre- and post-hMPV F antigen protein constructs were tested in a MIMIC system to assess the quality of the immunogenic response relative to controls. Control groups included: untreated control (no antigen without human skeletal muscle cells (w/o HSK)), reference antigen—RSV pre-F protein fused to ferritin nanoparticles (pre-F NP) and polio vaccine (IPOL).

### Material & Methods

Briefly, PBMCs were harvested via magnetic bead separation kit from 22 different human blood donors. Human dendritic cells (DCs) and B cells selected therefrom were added to and co-cultured with human skeletal muscle cells (HSKMC) and stimulated with either hMPV pre-F antigen protein (at 100 ng/ml or 500 ng/ml) or hMPV post F antigen protein (100 ng/ml). For B cell responses, following 14-day co-culture, supernatants we collected and analyzed for antibody specificity and function.

### Results:

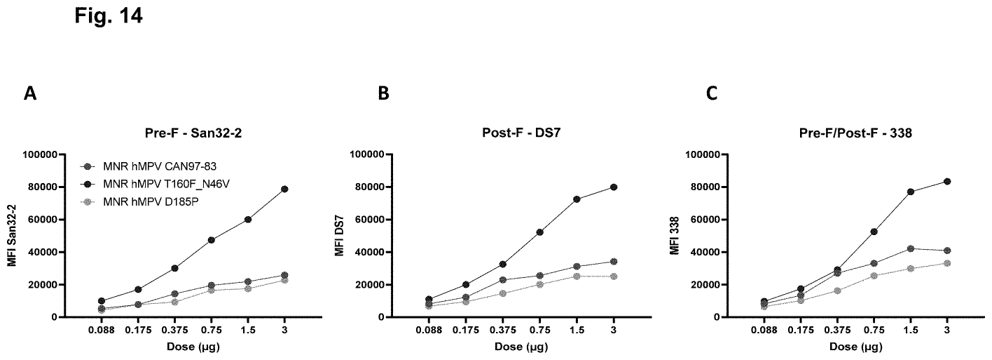

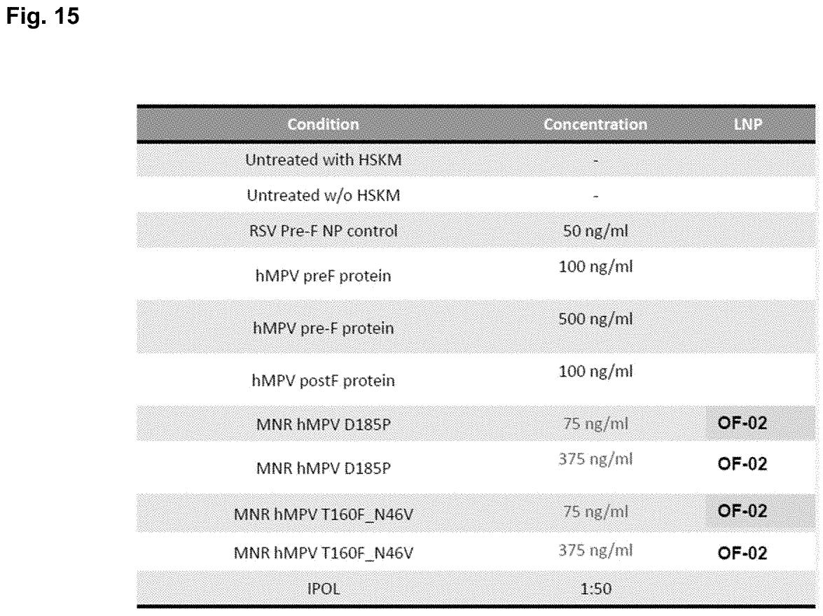

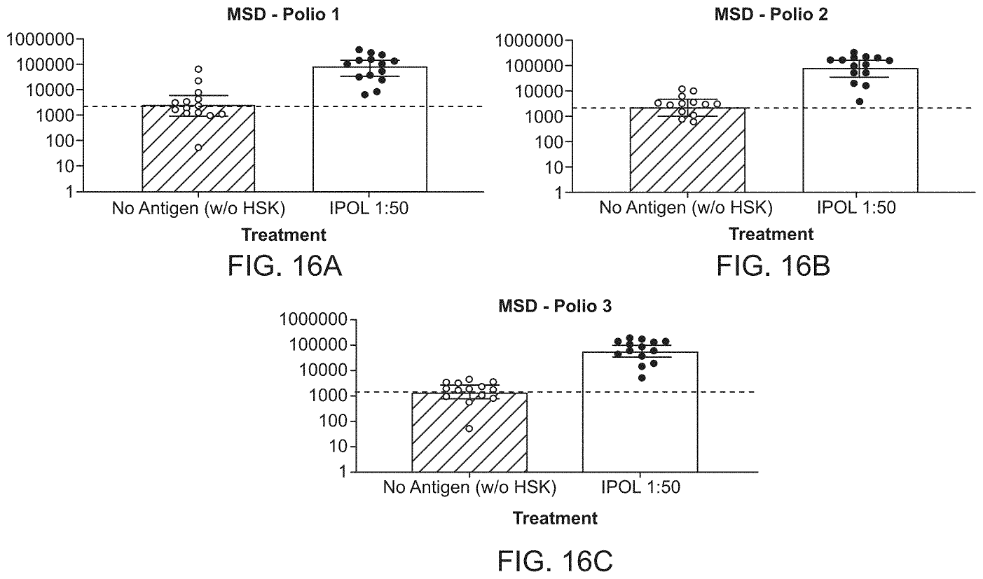

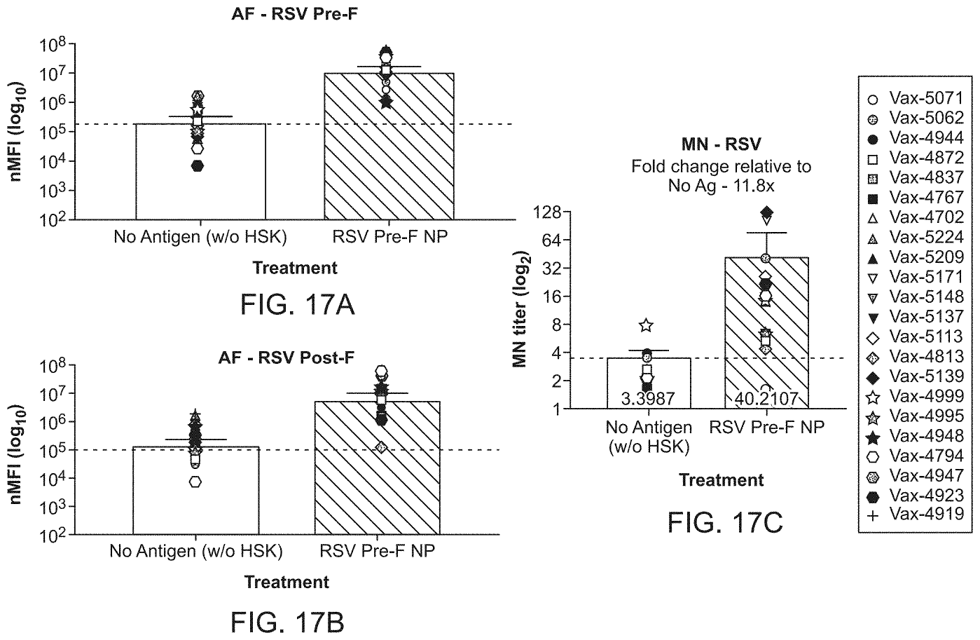

FIG. 15 depicts the MIMIC setup. Similar levels of expression were observed for T160F_N46V and D185P at doses of 75 ng/ml and 375 ng/ml for the shared pre-F/post-F epitope (FIG. 14, panels A-C). To confirm the activation MIMIC co-cultures, previously analyzed polio vaccine (IPOL) and antigen (RSV pre-F-NP) were used as positive controls. As shown in FIG. 16, panels A-C, the IPOL treatment in a 1:50 dilution elicited an antibody response to three Polio strains (Polio 1, 2, and 3) relative to untreated control. Similarly, 50 ng/ml RSV pre-F NP treatment of co-culture elicited an IgG specific antibody response to both RSV pre-F (FIG. 17, panel A) and RSV post-F (FIG. 17, panel B). Further, these antibodies were also functional as measured in an RSV neutralization assay (FIG. 17, panel C).

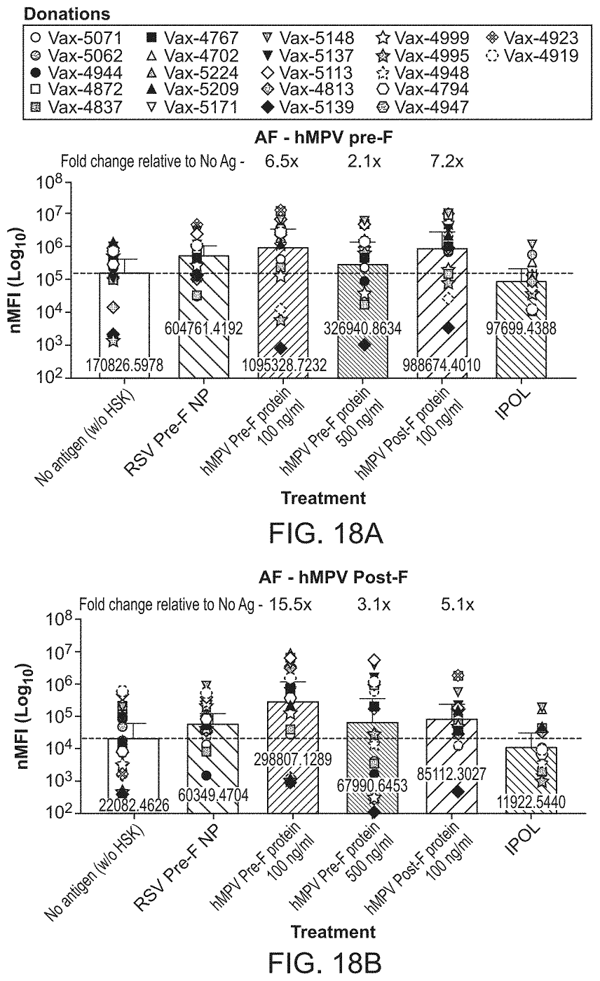

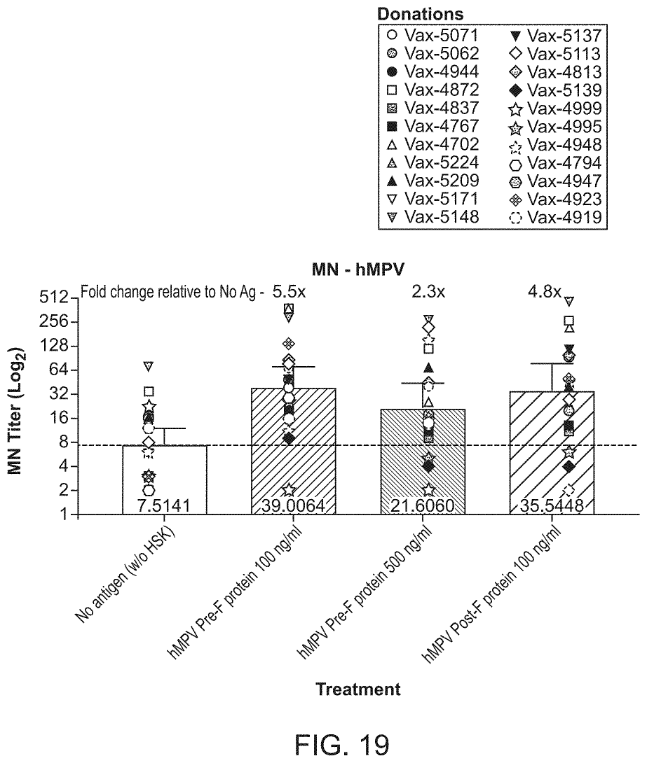

hMPV pre-F and post-F proteins showed high pre-F and post-F antibody responses (FIG. 18, panels A and B) and high neutralizing antibody titers (FIG. 19).

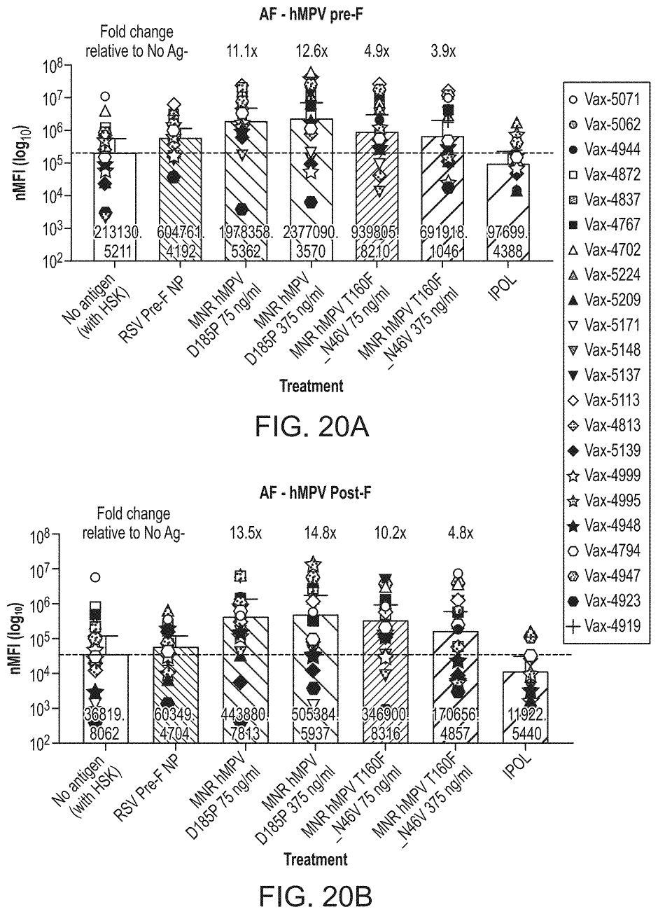

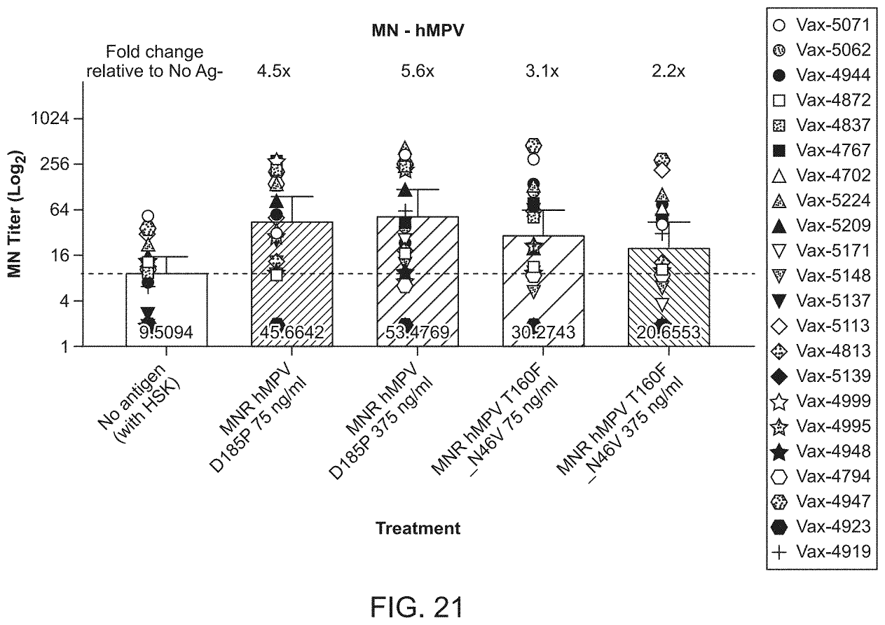

Supernatants from cocultures treated with experimental groups, hMPV pre-F antigen protein or hMPV post-F antigen protein elicited a robust IgG antibody response to both hMPV pre-F (FIG. 20, panel A) and hMPV post-F antigen (FIG. 20, panel B) relative to no antigen control. Also, these antibodies were functional as measured in a hMPV neutralization assay (FIG. 21). Antibodies from all three treatment groups bound to hMPV pre- and post-fusion F antigen and neutralized viral infectivity supporting the notion that pre- and post-fusion hMPV share neutralizing epitopes.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1. An antigenic human metapneumovirus (hMPV) prefusion F polypeptide, or a nucleic acid molecule that encodes the same, wherein said prefusion F polypeptide lacks a transmembrane domain and lacks a cytoplasmic tail, and comprises a human rhinovirus 3C (HRV-3C) protease cleavage site.

2. The F polypeptide or nucleic acid molecule of claim 1, wherein said prefusion F polypeptide further comprises
a F₀ cleavage site mutation comprising amino acid substitutions Q100R and S101R, replacing glutamine at amino acid position 100 of SEQ ID NO: 1 with arginine, and replacing serine at amino acid position 101 of SEQ ID NO: 1 with arginine;
a signal peptide;
at least one tag sequence that is optionally an 8×His tag and/or a Strep II tag, or a foldon domain;
an amino acid substitution replacing threonine at amino acid position 160 of SEQ ID NO: 1, and an amino acid substitution replacing asparagine at amino acid position 46 of SEQ ID NO: 1; or
at least 95% sequence identity to SEQ ID NO: 3 or comprises SEQ ID NO: 3.

3-6. (canceled)

7. An antigenic human metapneumovirus (hMPV) prefusion F polypeptide, or a nucleic acid molecule that encodes the same, wherein said prefusion F polypeptide lacks a transmembrane domain and lacks a cytoplasmic tail, and comprises:
an F₀ cleavage site mutation comprising amino acid substitutions Q100R and S101R; replacing glutamine at amino acid position 100 of SEQ ID NO: 1 with arginine, and replacing serine at amino acid position 101 of SEQ ID NO: 1 with arginine;
a human rhinovirus 3C (HRV-3C) protease cleavage site;
a heterologous signal peptide;
an 8×His tag and/or a Strep II tag; and
a foldon domain; or
an amino acid substitution replacing the wild-type amino acid at position 160 of SEQ ID NO: 1, and an amino acid substitution replacing the wild-type amino acid at position 46 of SEQ ID NO: 1; or
an amino acid substitution replacing threonine at amino acid position 160 of SEQ ID NO: 1, and an amino acid substitution replacing asparagine at amino acid position 46 of SEQ ID NO: 1.

8. (canceled)

9. (canceled)

10. The hMPV F polypeptide or nucleic acid molecule of claim 7, wherein said prefusion F polypeptide comprises
an amino acid substitution replacing the amino acid at position 160 with phenylalanine, tryptophan, tyrosine, valine, alanine, isoleucine or leucine optionally wherein said prefusion F polypeptide comprises an amino acid substitution replacing the amino acid at position 160 with phenylalanine;
an amino acid substitution replacing the amino acid at position 46 with valine, alanine, isoleucine, leucine, phenylalanine, tyrosine or proline optionally wherein said prefusion F polypeptide comprises an amino acid substitution replacing the amino acid at position 46 with valine;
at least 95% sequence identity to SEQ ID NO: 7;
an F₀ cleavage site mutation comprising amino acid substitutions Q100R and S101R, replacing glutamine at amino acid position 100 of SEQ ID NO: 1 with arginine, and replacing serine at amino acid position 101 of SEQ ID NO: 1 with arginine;
a signal peptide; or
at least one tag sequence that is optionally an 8×His tag and/or a Strep II tag, or a foldon domain.

11-18. (canceled)

19. An antigenic human metapneumovirus (hMPV) prefusion F polypeptide, or a nucleic acid molecule that encodes the same, wherein said prefusion F polypeptide lacks a transmembrane domain and lacks a cytoplasmic tail, and comprises:
an amino acid substitution T160F replacing threonine at amino acid position 160 of SEQ ID NO: 1 with phenylalanine, and an amino acid substitution N46V replacing asparagine at amino acid position 46 of SEQ ID NO: 1 with valine;
an F₀ cleavage site mutation comprising amino acid substitutions Q100R and S101R; replacing glutamine at amino acid position 100 of SEQ ID NO: 1 with arginine, and replacing serine at amino acid position 101 of SEQ ID NO: 1 with arginine;
a human rhinovirus 3C (HRV-3C) protease cleavage site;
a signal peptide;
an 8×His tag and/or a Strep II tag; and
a foldon domain.

20. The F polypeptide or nucleic acid molecule of claim 1, wherein the hMPV is A strain or B strain optionally wherein the hMPV is A1 subtype, A2 subtype, B1 subtype, or B2 subtype.

21. (canceled)

22. (canceled)

23. A human metapneumovirus (hMPV) F polypeptide, or a nucleic acid molecule that encodes the same, wherein said F polypeptide comprises at least 95% sequence identity to SEQ ID NO: 7 or comprises SEQ ID NO: 7, optionally wherein
the F polypeptide is a prefusion F polypeptide;
the F polypeptide is antigenic; or
the F polypeptide comprises amino acid substitution T160F replacing threonine at amino acid position 160 with phenylalanine, and amino acid substitution N46V replacing asparagine at amino acid position 46 with valine.

24-27. (canceled)

28. A nucleic acid molecule encoding the polypeptide of claim 23.

29. The nucleic acid molecule of claim 28, having at least 95% sequence identity to SEQ ID NO: 8 or comprising SEQ ID NO: 8, or having at least 95% sequence identity to SEQ ID NO: 18 or SEQ ID NO: 19, or comprising SEQ ID NO: 18 or SEQ ID NO: 19.

30. (canceled)

31. A pharmaceutical composition comprising the F polypeptide, or a nucleic acid molecule of claim 23.

32. The pharmaceutical composition of claim 31, comprising a vaccine.

33. A messenger RNA (mRNA) comprising an open reading frame (ORF) encoding the F polypeptide of claim 1.

34. A messenger RNA (mRNA) comprising an open reading frame (ORF) encoding a human metapneumovirus (hMPV) F polypeptide antigen, wherein the hMPV F polypeptide antigen comprises an amino acid sequence with at least 95% identity to SEQ ID NO: 11 or consists of an amino acid sequence of SEQ ID NO: 11.

35. The mRNA of claim 33, wherein the hMPV F polypeptide antigen comprises:
(a) a pre-fusion F polypeptide;
(b) the ORF is codon optimized; or
(c) at least one 5′ untranslated region (5′ UTR), at least one 3′ untranslated region (3′ UTR), and at least one polyadenylation (poly(A)) sequence.

36. (canceled)

37. (canceled)

38. The mRNA of any one of claim 33, wherein the mRNA comprises at least one chemical modification, optionally wherein
at least 20%, at least 30%, at least 40%, at least 50%, at least 60%, at least 70%, at least 80%, at least 85%, at least 90%, at least 95%, or 100% of the uracil nucleotides in the mRNA are chemically modified, or
wherein at least 20%, at least 30%, at least 40%, at least 50%, at least 60%, at least 70%, at least 80%, at least 85%, at least 90%, at least 95%, or 100% of the uracil nucleotides in the ORF are chemically modified.

39. (canceled)

40. (canceled)

41. The mRNA of claim 38, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine, optionally wherein the chemical modification is selected from the group consisting of pseudouridine, N1-methylpseudouridine, 5-methylcytosine, 5-methoxyuridine, and a combination thereof or wherein the chemical modification is N1-methylpseudouridine.

42. (canceled)

43. (canceled)

44. The mRNA of claim 33, wherein the mRNA is formulated in a lipid nanoparticle (LNP).

45. The mRNA of claim 44, wherein the LNP comprises at least one cationic lipid
optionally wherein the LNP cationic lipid is biodegradable, is not biodegradable, is cleavable, and/or is not cleavable, or
wherein the cationic lipid is selected from the group consisting of OF-02, cKK-E10, GL-HEPES-E3-E10-DS-3-E18-1, GL-HEPES-E3-E12-DS-4-E10, and GL-HEPES-E3-E12-DS-3-E14 optionally wherein the cationic lipid is cKK-E10 or the cationic lipid is GL-HEPES-E3-E12-DS-4-E10.

46-52. (canceled)

53. The mRNA of claim 44, wherein the LNP further comprises a polyethylene glycol (PEG) conjugated (PEGylated) lipid, a cholesterol-based lipid, and a helper lipid,
optionally wherein the PEGylated lipid is dimyristoyl-PEG2000 (DMG-PEG2000) or 2-[(polyethylene glycol)-2000]-N,N-ditetradecylacetamide (ALC-0159), the cholesterol-based lipid is cholesterol, or the helper lipid is 1,2-dioleoyl-SN-glycero-3-phosphoethanolamine (DOPE) or 1,2-di stearoyl-sn-glycero-3-phosphocholine (DSPC).

54. The mRNA of claim 44, wherein the LNP comprises:
a cationic lipid at a molar ratio of 35% to 55%,
a polyethylene glycol (PEG) conjugated (PEGylated) lipid at a molar ratio of 0.25% to 2.75%,
a cholesterol-based lipid at a molar ratio of 20% to 45%, and
a helper lipid at a molar ratio of 5% to 35%,
wherein all of the molar ratios are relative to the total lipid content of the LNP; or
a cationic lipid at a molar ratio of 40%,
a PEGylated lipid at a molar ratio of 1.5%,
a cholesterol-based lipid at a molar ratio of 28.5%, and
a helper lipid at a molar ratio of 30%.

55-58. (canceled)

59. The mRNA of claim 44, wherein the LNP comprises:
GL-HEPES-E3-E12-DS-4-E10 or cKK-E10 at a molar ratio of 40%,
DMG-PEG2000 at a molar ratio of 1.5%,
cholesterol at a molar ratio of 28.5%, and
DOPE at a molar ratio of 30%.

60. (canceled)

61. The mRNA of claim 44, wherein the LNP has an average diameter of 30 nm to 200 nm or has an average diameter of 80 nm to 150 nm.

62. (canceled)

63. A pharmaceutical composition comprising the mRNA of claim 33.

64. The pharmaceutical composition of claim 63, comprising a vaccine.

65. A method of eliciting an immune response to hMPV or protecting a subject against hMPV infection, comprising administering the vaccine of claim 32 to a subject.

66. The method of claim 65, wherein:
the subject has a comparable serum concentration of neutralizing antibodies against hMPV after administration of the vaccine, relative to a subject that is administered a protein hMPV vaccine; the protein hMPV vaccine is co-administered with an adjuvant; or the vaccine increases the serum concentration of neutralizing antibodies in a subject with pre-existing hMPV immunity.

67-70. (canceled)

71. A method of eliciting an immune response or of preventing an hMPV infection or reducing one or more symptoms of an hMPV infection in a subject in need thereof, comprising administering to the subject, optionally intramuscularly, intranasally, intravenously, subcutaneously, or intradermally, a prophylactically effective amount of the F polypeptide or nucleic acid molecule of claim 1.

72-74. (canceled)

75. A kit comprising a container comprising a single-use or multi-use dosage of the F polypeptide or nucleic acid molecule of claim 1, optionally wherein the container is a vial or a pre-filled syringe or injector.

76. A vaccine comprising a human metapneumovirus (hMPV) F polypeptide antigen or a nucleic acid molecule that encodes the same, wherein the F polypeptide comprises an amino acid sequence having at least 95% identity to SEQ ID NO: 7 or consisting of an amino acid sequence of SEQ ID NO: 7, optionally wherein the hMPV F polypeptide is a pre-fusion F polypeptide.

77. (canceled)

78. A method of eliciting an immune response to hMPV or protecting a subject against hMPV infection or of preventing an hMPV infection or reducing one or more symptoms of an hMPV infection, comprising administering the vaccine of claim 76 to a subject, optionally wherein the vaccine:
increases the serum concentration of neutralizing antibodies, and wherein the subject has pre-existing hMPV immunity
is co-administered with an adjuvant;
is administered in combination with an additional vaccine, optionally wherein the additional vaccine is a respiratory syncytial virus (RSV) vaccine or an influenza vaccine;
is administered to a human subject, optionally wherein the human subject is an infant, a toddler, or an older adult; or
is administered to the subject intramuscularly, intranasally, intravenously, subcutaneously, intradermally, or in a prophylactically effective amount of the vaccine.

79-91. (canceled)

92. An expression vector encoding the F polypeptide or the nucleic acid molecule of claim 1.

93. A cell comprising the expression vector of claim 92.


SEQUENCE LISTING (28 sequences)

SEQ ID NO: 1 - protein, 539 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN

SEQ ID NO: 2 - RNA, 1620 nt, synthetic construct
  source 1..1620: mol_type = other RNA
atgtcttggaaagtggtgatcattttttcattgctaataacacctcaacacggtcttaaagagagctacctagaagaatcatgtagcactataactgagggatatcttagtgttctgaggacaggttggtataccaacgtttttacattagaggtgggtgatgtagaaaaccttacatgttctgatggacctagcctaataaaaacagaattagatctgaccaaaagtgcactaagagagctcaaaacagtctctgctgaccaattggcaagagaggaacaaattgagaatcccagacaatctaggtttgttctaggagcaatagcactcggtgttgcaacagcagctgcagtcacagcaggtgttgcaattgccaaaaccatccggcttgagagtgaagtcacagcaattaagaatgccctcaaaacgaccaatgaagcagtatctacattggggaatggagttcgagtgttggcaactgcagtgagagagctgaaagactttgtgagcaagaatttaactcgtgcaatcaacaaaaacaagtgcgacattgatgacctaaaaatggccgttagcttcagtcaattcaacagaaggtttctaaatgttgtgcggcaattttcagacaatgctggaataacaccagcaatatctttggacttaatgacagatgctgaactagccagggccgtttctaacatgccgacatctgcaggacaaataaaattgatgttggagaaccgtgcgatggtgcgaagaaaggggttcggaatcctgataggggtctacgggagctccgtaatttacatggtgcagctgccaatctttggcgttatagacacgccttgctggatagtaaaagcagccccttcttgttccgaaaaaaagggaaactatgcttgcctcttaagagaagaccaagggtggtattgtcagaatgcagggtcaactgtttactacccaaatgagaaagactgtgaaacaagaggagaccatgtcttttgcgacacagcagcgggaattaatgttgctgagcaatcaaaggagtgcaacatcaacatatccactacaaattacccatgcaaagtcagcacaggaagacatcctatcagtatggttgcactgtctcctcttggggctctggttgcttgctacaaaggagtaagctgttccattggcagcaacagagtagggatcatcaagcagctgaacaagggttgctcctatataaccaaccaagatgcagacacagtgacaatagacaacactgtatatcagctaagcaaagttgagggtgaacagcatgttataaaaggcagaccagtgtcaagcagctttgatccaatcaagtttcctgaagatcaattcaatgttgcacttgaccaagtttttgagaacattgaaaacagccaggccttggtagatcaatcaaacagaatcctaagcagtgcagagaaagggaatactggcttcatcattgtaataattctaattgctgtccttggctctagcatgatcctagtgagcatcttcattataatcaagaaaacaaagaaaccaacgggagcacctccagagctgagtggtgtcacaaacaatggcttcataccacataattag

SEQ ID NO: 3 - protein, 551 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRRRRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGGGSGYIPEAPRDGQAYVRKDGEWVLLSTFLGRSLEVLFQGPGHHHHHHHHSAWSHPQFEK

SEQ ID NO: 4 - RNA, 1656 nt, synthetic construct
  source 1..1656: mol_type = other RNA
atgagttggaaggtggtgattatcttctccctgctgattacaccacaacatggactgaaagagtcctacttggaggagtcctgtagcaccatcacagagggctacctgtctgtgctgaggacaggctggtacaccaatgtgttcaccttggaggtgggagatgtggagaacctgacttgttctgatggaccatccctgattaagacagaactggacctgaccaagtctgccctgagggaactgaaaacagtgtctgctgaccaacttgccagggaggaacagattgagaacccaaggaggaggaggtttgtgctgggagccattgccctgggagtggctacagcagcagcagtgacagcaggagtggctattgccaagaccatcagattggagtctgaggtgacagccatcaagaatgccctgaaaaccaccaatgaggctgtgagcaccctgggcaatggagtgagggtgctggctacagcagtgagggaactgaaagactttgtgagcaagaacctgaccagggctatcaacaagaacaagtgtgacatcgatgacctgaaaatggctgtgtccttcagccagttcaacaggaggttcctgaatgtggtgagacagttctctgacaatgctggcatcacacctgccatctccctggacctgatgacagatgctgaactggcaagggctgtgagcaatatgccaacctctgctggacaaatcaaactgatgttggagaacagggctatggtgaggaggaagggctttggcatcctgattggagtctatggctcctctgtgatttatatggtccaacttccaatctttggagtgattgacacaccatgttggattgtgaaggctgccccatcctgttctgagaagaagggcaactatgcctgtctgctgagggaggaccagggctggtattgtcagaatgctggcagcacagtctactacccaaatgagaaggactgtgagaccaggggagaccatgtgttctgtgacacagcagcaggcatcaatgtggctgaacagagcaaggagtgtaacatcaacatcagcaccaccaactacccatgtaaggtgagcacaggcagacacccaatcagtatggtggctctgagcccactgggagccctggtggcttgttacaagggagtgtcctgtagcattggcagcaacagggtgggcatcatcaagcaacttaacaagggctgttcctacatcaccaaccaggatgctgacacagtgaccattgacaacacagtctaccaacttagcaaggtggagggagaacagcatgtgattaagggcagacctgtgtcctcctcctttgacccaatcaagtttcctgaggaccagttcaatgtggctctggaccaggtgtttgagaacattgagaacagccaggctctggtggaccagagcaacaggattctgtcctctgctgagaagggcaacacaggaggaggaggctctggctacatccctgaggctccaagggatggacaagcctatgtgaggaaggatggagagtgggtgctgctgagcaccttcctgggcaggtccttggaggtgctgttccagggacctggacaccaccaccaccaccaccaccactctgcctggagccacccacagtttgagaagtaa

SEQ ID NO: 5 - protein, 551 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRRRRFVLGAIALGVATAAAVTAGVAIAKTIRLESEVTAIKNALKTTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGGGSGYIPEAPRDGQAYVRKDGEWVLLSTFLGRSLEVLFQGPGHHHHHHHHSAWSHPQFEK

SEQ ID NO: 6 - RNA, 1656 nt, synthetic construct
  source 1..1656: mol_type = other RNA
atgagttggaaggtggtgattatcttctccctgctgattacaccacaacatggactgaaagagtcctacttggaggagtcctgtagcaccatcacagagggctacctgtctgtgctgaggacaggctggtacaccaatgtgttcaccttggaggtgggagatgtggagaacctgacttgttctgatggaccatccctgattaagacagaactggacctgaccaagtctgccctgagggaactgaaaacagtgtctgctgaccaacttgccagggaggaacagattgagaacccaaggaggaggaggtttgtgctgggagccattgccctgggagtggctacagcagcagcagtgacagcaggagtggctattgccaagaccatcagattggagtctgaggtgacagccatcaagaatgccctgaaaaccaccaatgaggctgtgagcaccctgggcaatggagtgagggtgctggctacagcagtgagggaactgaaagactttgtgagcaagaacctgaccagggctatcaacaagaacaagtgtgacatccctgacctgaaaatggctgtgtccttcagccagttcaacaggaggttcctgaatgtggtgagacagttctctgacaatgctggcatcacacctgccatctccctggacctgatgacagatgctgaactggcaagggctgtgagcaatatgccaacctctgctggacaaatcaaactgatgttggagaacagggctatggtgaggaggaagggctttggcatcctgattggagtctatggctcctctgtgatttatatggtccaacttccaatctttggagtgattgacacaccatgttggattgtgaaggctgccccatcctgttctgagaagaagggcaactatgcctgtctgctgagggaggaccagggctggtattgtcagaatgctggcagcacagtctactacccaaatgagaaggactgtgagaccaggggagaccatgtgttctgtgacacagcagcaggcatcaatgtggctgaacagagcaaggagtgtaacatcaacatcagcaccaccaactacccatgtaaggtgagcacaggcagacacccaatcagtatggtggctctgagcccactgggagccctggtggcttgttacaagggagtgtcctgtagcattggcagcaacagggtgggcatcatcaagcaacttaacaagggctgttcctacatcaccaaccaggatgctgacacagtgaccattgacaacacagtctaccaacttagcaaggtggagggagaacagcatgtgattaagggcagacctgtgtcctcctcctttgacccaatcaagtttcctgaggaccagttcaatgtggctctggaccaggtgtttgagaacattgagaacagccaggctctggtggaccagagcaacaggattctgtcctctgctgagaagggcaacacaggaggaggaggctctggctacatccctgaggctccaagggatggacaagcctatgtgaggaaggatggagagtgggtgctgctgagcaccttcctgggcaggtccttggaggtgctgttccagggacctggacaccaccaccaccaccaccaccactctgcctggagccacccacagtttgagaagtaa

SEQ ID NO: 7 - protein, 551 aa, synthetic construct
MSWKVVIIFSLLITPQHGLKESYLEESCSTITEGYLSVLRTGWYTVVFTLEVGDVENLTCSDGPSLIKTELDLTKSALRELKTVSADQLAREEQIENPRRRRFVLGAIALGVATAAAVTAGVAIAKTIRLESEVTAIKNALKTTNEAVSTLGNGVRVLAFAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGGGSGYIPEAPRDGQAYVRKDGEWVLLSTFLGRSLEVLFQGPGHHHHHHHHSAWSHPQFEK

SEQ ID NO: 8 - RNA, 1656 nt, synthetic construct
  source 1..1656: mol_type = other RNA
atgagttggaaggtggtgattatcttctccctgctgattacaccacaacatggactgaaagagtcctacttggaggagtcctgtagcaccatcacagagggctacctgtctgtgctgaggacaggctggtacacagtggtgttcaccttggaggtgggagatgtggagaacctgacttgttctgatggaccatccctgattaagacagaactggacctgaccaagtctgccctgagggaactgaaaacagtgtctgctgaccaacttgccagggaggaacagattgagaacccaaggaggaggaggtttgtgctgggagccattgccctgggagtggctacagcagcagcagtgacagcaggagtggctattgccaagaccatcagattggagtctgaggtgacagccatcaagaatgccctgaaaaccaccaatgaggctgtgagcaccctgggcaatggagtgagggtgctggcttttgctgtgagggaactgaaagactttgtgagcaagaacctgaccagggctatcaacaagaacaagtgtgacattgatgacctgaaaatggctgtgtccttcagccagttcaacaggaggttcctgaatgtggtgagacagttctctgacaatgctggcatcacacctgccatctccctggacctgatgacagatgctgaactggcaagggctgtgagcaatatgccaacctctgctggacaaatcaaactgatgttggagaacagggctatggtgaggaggaagggctttggcatcctgattggagtctatggctcctctgtgatttatatggtccaacttccaatctttggagtgattgacacaccatgttggattgtgaaggctgccccatcctgttctgagaagaagggcaactatgcctgtctgctgagggaggaccagggctggtattgtcagaatgctggcagcacagtctactacccaaatgagaaggactgtgagaccaggggagaccatgtgttctgtgacacagcagcaggcatcaatgtggctgaacagagcaaggagtgtaacatcaacatcagcaccaccaactacccatgtaaggtgagcacaggcagacacccaatcagtatggtggctctgagcccactgggagccctggtggcttgttacaagggagtgtcctgtagcattggcagcaacagggtgggcatcatcaagcaacttaacaagggctgttcctacatcaccaaccaggatgctgacacagtgaccattgacaacacagtctaccaacttagcaaggtggagggagaacagcatgtgattaagggcagacctgtgtcctcctcctttgacccaatcaagtttcctgaggaccagttcaatgtggctctggaccaggtgtttgagaacattgagaacagccaggctctggtggaccagagcaacaggattctgtcctctgctgagaagggcaacacaggaggaggaggctctggctacatccctgaggctccaagggatggacaagcctatgtgaggaaggatggagagtgggtgctgctgagcaccttcctgggcaggtccttggaggtgctgttccagggacctggacaccaccaccaccaccaccaccactctgcctggagccacccacagtttgagaagtaa

SEQ ID NO: 9 - protein, 539 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN

SEQ ID NO: 10 - skipped

SEQ ID NO: 11 - protein, 539 aa, synthetic construct
MSWKVVIIFSLLITPQHGLKESYLEESCSTITEGYLSVLRTGWYTVVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLAFAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN

SEQ ID NO: 12-14 - skipped (3 entries)

SEQ ID NO: 15 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 16 - RNA, 11 nt, synthetic construct
  source 1..11: mol_type = other RNA
gggatcctacc

SEQ ID NO: 17 - RNA, 1620 nt, synthetic construct
  source 1..1620: mol_type = other RNA
atgtcttggaaagtcgtcatcatcttctctctgctgatcaccccacaacacggcctgaaggaatcttatctggaagagtcctgctccacaatcacagagggctacctgagcgtgctgagaaccggctggtacaccaacgtgttcactctggaggtgggcgacgtggagaacctgacttgtagtgacggcccctccctgatcaagactgagctggacctgacaaagagtgcactgagagaactcaagactgtgtccgcagaccagctggcccgcgaggagcagatcgaaaatcctagacagtcaaggttcgtcctgggagccattgctctgggagttgctacagctgccgctgtgaccgcaggggtggctattgctaaaaccatcaggctggagtccgaagtgacagcaatcaagaatgccctgaagaccaccaacgaggcagtctccacactgggcaatggagtgagggtgctggcaaccgccgtgagggagctgaaggacttcgtgtccaagaacctgaccagggctatcaacaaaaacaagtgcgacatccccgatctgaagatggcagttagcttttcccagtttaaccggagattcctgaatgtggttagacagttcagcgacaacgccgggatcaccccagctatttccctggacctgatgactgatgccgagctggcacgggctgtgtccaatatgcccaccagcgctgggcagattaagctgatgctggagaatcgggcaatggtgagaaggaaggggtttggcatcctgatcggcgtgtacgggtcctccgtgatctacatggtgcagctgcctatttttggagtgattgatacaccctgctggatcgttaaagcagcacccagctgctccgagaagaagggcaattacgcctgtctgctgcgggaggaccaggggtggtactgccagaacgccggctccacagtgtattaccccaatgaaaaggactgcgagacaaggggagaccacgtgttctgcgacactgccgctgggattaatgtggccgagcagagcaaggagtgcaacatcaacatttccaccacaaactacccctgcaaggtgagcaccggcaggcaccctatctccatggtggccctgtctcccctgggagctctggtggcttgctacaagggagtgagctgtagcatcgggtccaatagagtcgggattatcaagcagctgaataagggctgcagctatattaccaaccaggatgccgatactgtgactattgacaacacagtgtatcagctgtcaaaggtggaaggcgaacagcatgtgatcaaaggacggcccgtcagcagctcctttgaccctatcaaattccccgaagaccagtttaacgtggcactggaccaggttttcgaaaatattgagaattctcaggccctggtggaccagtctaaccggatcctctcctccgccgagaagggaaatacaggctttattatcgtgatcatcctgatcgcagtgctgggatccagtatgatcctggtctccattttcatcatcattaagaagaccaagaaacccactggcgcaccacctgaactgagcggcgtgactaacaatggctttatccctcacaattga

SEQ ID NO: 18 - RNA, 1620 nt, synthetic construct
  source 1..1620: mol_type = other RNA
atgagctggaaggttgtgattattttctctctgctgattactccacagcacggcctgaaggagtcctacctggaggagtcctgttctactatcactgaggggtatctctctgtgctgcggacagggtggtatacagtggtgttcaccctggaggttggcgatgtggagaatctgacttgcagcgatggcccttctctgatcaagaccgagctggatctgacaaaaagcgccctcagagaactgaaaaccgtgtccgccgatcagctggcaagggaggagcagatcgagaacccacggcagagcaggtttgtgctgggcgctatcgctctgggcgtggccactgcagctgctgtcactgcaggggtcgcaatcgctaagactatcagactggaatccgaggtgaccgccattaagaatgccctgaagactaccaacgaggctgtgtccactctgggaaacggagtgagggtcctggccttcgcagtgagggagctgaaggattttgtgtcaaagaaccttacacgggccatcaacaagaataagtgcgatatcgatgacctgaagatggccgtgtccttctcccagttcaaccggcgctttctgaatgtggtgcgccagttttccgacaacgctggaatcacccctgctatcagcctggacctcatgaccgacgccgaactcgcaagggccgtttctaacatgcctacatccgctggacagattaagctgatgctggagaatagagcaatggtgaggagaaagggattcggcatcctgattggcgtgtacggatctagcgtgatctacatggtgcagctgccgatcttcggcgtgatcgatactccttgttggatcgtcaaggccgccccttcctgctccgagaagaagggcaattacgcttgtctgctgcgggaggaccagggctggtattgccagaacgccgggtctacagtgtactatcctaacgagaaggattgcgagaccagaggcgaccacgttttctgtgatacagccgccggaatcaatgtcgcagagcagtctaaggagtgcaacatcaatatctctacaaccaattacccatgtaaggtgagcactggacggcaccctatcagtatggtggctctgagcccactgggggcactggtggcttgctacaagggggtgagctgcagtatcggcagtaacagagtgggcattatcaagcagctgaacaaagggtgctcttatattacaaaccaggatgcagatactgtgaccatcgacaacactgtgtaccagctgtccaaggtggagggggagcagcatgtgatcaaagggagacccgtctcttcttctttcgatcccatcaagttccctgaagaccagttcaatgttgccctggaccaggttttcgagaacatcgaaaatagccaggccttggtcgatcaatccaacaggatcctgagcagcgcagagaaagggaacactggcttcatcatcgtgatcattctgatcgccgtgctggggagcagtatgattctggtgtccattttcatcatcatcaagaagaccaagaagcctacaggagcaccccctgagctgagcggagtgaccaacaacggctttatccctcacaactga

SEQ ID NO: 19 - RNA, 1620 nt, synthetic construct
  source 1..1620: mol_type = other RNA
atgagctggaaggttgtgattattttctctctgctgattactccacagcacggcctgaaggagtcctacctggaggagtcctgttctactatcactgaggggtatctctctgtgctgcggacagggtggtatacagtggtgttcaccctggaggttggcgatgtggagaatctgacttgcagcgatggcccttctctgatcaagaccgagctggatctgacaaaaagcgccctcagagaactgaaaaccgtgtccgccgatcagctggcaagggaggagcagatcgagaacccacggcagagcaggtttgtgctgggcgctatcgctctgggcgtggccactgcagctgctgtcactgcaggggtcgcaatcgctaagactatcagactggaatccgaggtgaccgccattaagaatgccctgaagactaccaacgaggctgtgtccactctgggaaacggagtgagggtcctggccttcgcagtgagggagctgaaggattttgtgtcaaagaaccttacacgggccatcaacaagaataagtgcgatatcgatgacctgaagatggccgtgtccttctcccagttcaaccggcgctttctgaatgtggtgcgccagttttccgacaacgctggaatcacccctgctatcagcctggacctcatgaccgacgccgaactcgcaagggccgtttctaacatgcctacatccgctggacagattaagctgatgctggagaatagagcaatggtgaggagaaagggattcggcatcctgattggcgtgtacggatctagcgtgatctacatggtgcagctgccgatcttcggcgtgatcgatactccttgttggatcgtcaaggccgccccttcctgctccgagaagaagggcaattacgcttgtctgctgcgggaggaccagggctggtattgccagaacgccgggtctacagtgtactatcctaacgagaaggattgcgagaccagaggcgaccacgttttctgtgatacagccgccggaatcaatgtcgcagagcagtctaaggagtgcaacatcaatatctctacaaccaattacccatgtaaggtgagcactggacggcaccctatcagtatggtggctctgagcccactgggggcactggtggcttgctacaagggggtgagctgcagtatcggcagtaacagagtgggcattatcaagcagctgaacaaagggtgctcttatattacaaaccaggatgcagatactgtgaccatcgacaacactgtgtaccagctgtccaaggtggagggggagcagcatgtgatcaaagggagacccgtctcttcttctttcgatcccatcaagttccctgaagaccagttcaatgttgccctggaccaggttttcgagaacatcgaaaatagccaggccttggtcgatcaatccaacaggatcctgagcagcgcagagaaagggaacactggcttcatcatcgtgatcattctgatcgccgtgctggggagcagtatgattctggtgtccattttcatcatcatcaagaagaccaagaagcctacaggagcaccccctgagctgagcggagtgaccaacaacggctttatccctcacaactaa

SEQ ID NO: 20 - protein, 551 aa, synthetic construct; identical to SEQ ID NO: 5

SEQ ID NO: 21 - protein, 551 aa, synthetic construct; identical to SEQ ID NO: 7

SEQ ID NO: 22 - protein, 24 aa, synthetic construct
GFIIVIILIAVLGSSMILVSIFII

SEQ ID NO: 23 - protein, 26 aa, synthetic construct
IKKTKKPTGAPPELSGVTNNGFIPHN

SEQ ID NO: 24 - protein, 7 aa, synthetic construct
ENPRRRR

SEQ ID NO: 25 - protein, 5 aa, synthetic construct
GGGGS

SEQ ID NO: 26 - protein, 27 aa, synthetic construct
GYIPEAPRDGQAYVRKDGEWVLLSTFL

SEQ ID NO: 27 - protein, 8 aa, synthetic construct
HHHHHHHH

SEQ ID NO: 28 - protein, 10 aa, synthetic construct
SAWSHPQFEK